(19) United States
(12) Patent Application Publication
(10) Pub. No.: US 2022/0022747 A1
(43) Pub. Date: Jan. 27, 2022

(54) PERSONAL THERMAL DATA COLLECTION AND AGGREGATION SYSTEM AND METHOD

(71) Applicant: Vidal Malfavon, Norwalk, CA (US)

(72) Inventor: Vidal Malfavon, Norwalk, CA (US)

(21) Appl. No.: 16/939,023

(22) Filed: Jul. 26, 2020

(51) Int. Cl. A61B 5/00; A61B 5/01; A61B 5/0205; G06F 17/18

(52) U.S. Cl. CPC A61B 5/0008; A61B 5/01; A61B 5/0077; G06F 17/18; A61B 5/6801; A61B 5/7264; A61B 5/02055

(57) ABSTRACT

A thermal image data acquisition device is described herein, comprising: at least one wireless transceiver device, adapted to transmit and receive digital data; at least one infrared camera device, adapted to acquire one or more thermal images of an owner of the thermal image data acquisition device, wherein each of the one or more thermal images records an internal body temperature of the owner; a processor communicatively coupled to the at least one wireless transceiver device and the at least one infrared camera device; and a memory operatively connected to the processor, wherein the memory stores computer-executable instructions that, when executed by the processor, causes the at least one processor to execute a method that comprises: acquiring, via the at least one infrared camera device, the one or more thermal images within a thermal imaging application executing on the processor; and processing the acquired one or more thermal images in accordance with one or more processing instructions, wherein the one or more processing instructions generates one or more processed thermal image files; transmitting each of the one or more processed thermal image files of the owner; and making a determination as to whether a pandemic situation exists within a specific geographical area based on the recorded internal body temperature of the owner as stored in the one or more processed thermal data image files of the owner.

### BACKGROUND OF THE INVENTION

### Technical Field

The embodiments described herein relate generally to health management, and more specifically to systems, methods, and modes for a personal mobile electronic communications device that can automatically record a thermal image of a user-owner of the personal mobile electronic communications device, as well as to thermally image others by the same personal mobile electronic communications device.

### Background Art

Currently, the world is in the grip of a pandemic caused by the Coronavirus (virus). That is, at least several million people have been exposed to the virus, over a million people have become sick, over 300,000 people have died, and while there appear to be protocols that can mitigate and/or reduce the spread of the virus, and vaccines are being developed to prevent coronavirus infections from occurring, no medical or governmental authority is stating that we are past the point of danger. Indeed, many such medical experts and government leaders are warning of second waves of infections, as well as a surge associated with the cold winter months that could occur later this year.

There are several aspects of the virus that makes it particularly troubling. First is its rate of infection, or how easy it is to become infected and affected by the virus. The rate of infection, or ROI, is roughly about 2.1. This means for every single person infected by the virus, absent preventative measures, 2.1 other people will become sick. Secondly, the length of time between introduction and the appearance of symptoms can be up to 14 days. Third, many people can acquire the virus, but even after the 14 days “gestation” period, can be asymptomatic, meaning these people are unaffected and show no symptoms or only mild symptoms (which can be easily confused with a common cold or mild flu). Fourth, the severity of the illness brought about the virus is particularly problematic. For those that do catch the virus, and are particularly vulnerable to it, the affects can be devastating. Aside from the normal flu-like symptoms that people experience, lungs fill with fluid and it becomes exceedingly difficult to breathe. Ventilators are needed to assist many people in breathing. The mortality rate of patients who need assisted breathing with ventilators is extremely high, and ranges from 50-90%. According to competent medical authorities, a patient that needs the use of a ventilator is “unbelievably” sick. And in addition, for many people, complete recovery does not occur, and/or can take a significant amount of time, and can leave them with complications such as impaired cognitive ability, speech impediments, muscular and lung problems, among other issues.

In view of such dire statistics and mortality rates, most governments of the world instituted “preventative measures” to slow down the rate of transmission of the virus. In affect, people all over the world were requested to “stay in place.” That is, stay home, close businesses, stop traveling, and go out as little as possible. The world economy, as a result, has literally coming to a screeching halt. Oil prices have dropped to unheard of prices—for at least one day, traders were paying others to take the oil before it was delivered to them. Massive unemployment has occurred not only in the United States, but in many other countries. The economic affects have been, in short, overwhelming, and unbelievable. Nothing like this has ever happened so quickly to any economy ever.

The “preventative measures” described above included the “stay in place” orders, business shutdowns, and many other such measures. However, most rationale people have begun to realize no country or people can “stay in place” forever. It is a simple fact of nature that the virus is not going away, and that the “cure could be worse than the disease.” In addition, as time passes and scientists, doctors, and governments learn more about how the virus behaves, it has become very apparent that this virus affects different people in vastly different ways. That is, certain people and/or groups of people exhibit “comorbidities” that make them particularly susceptible to the affects of the virus, while many others, indeed the grand majority of others, will experience little to no symptoms, let alone be in danger of dying.

Nonetheless, no one wants to experience what the majority of the world went through from March of 2020 through the late Spring. To that end, it has become apparent that one of the first indicators that a person is infected with the virus is a rise in body temperature.

Accordingly, a need has arisen for systems, methods, and modes for a personal mobile electronic communications device that can automatically record a thermal image of a user-owner of the personal mobile electronic communications device, as well as to thermally image others by the same personal mobile electronic communications device.

### SUMMARY

It is an object of the embodiments to substantially solve at least the problems and/or disadvantages discussed above, and to provide at least one or more of the advantages described below.

It is therefore a general aspect of the embodiments to provide systems, methods, and modes for a personal electronic communications device that can automatically record a thermal image of a user-owner of the personal electronic communications device, as well as to thermally image others by the same personal electronic communications device that will obviate or minimize problems of the type previously described.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Further features and advantages of the aspects of the embodiments, as well as the structure and operation of the various embodiments, are described in detail below with reference to the accompanying drawings. It is noted that the aspects of the embodiments are not limited to the specific embodiments described herein. Such embodiments are presented herein for illustrative purposes only. Additional embodiments will be apparent to persons skilled in the relevant art(s) based on the teachings contained herein.

According to a first aspect of the embodiments, a thermal image data acquisition device is provided comprising: at least one wireless transceiver device, adapted to transmit and receive digital data; at least one infrared camera device, adapted to acquire one or more thermal images of an owner of the thermal image data acquisition device, wherein each of the one or more thermal images records an internal body temperature of the owner; a processor communicatively coupled to the at least one wireless transceiver device and the at least one infrared camera device; and a memory operatively connected to the processor, wherein the memory stores computer-executable instructions that, when executed by the processor, causes the at least one processor to execute a method that comprises: acquiring, via the at least one infrared camera device, the one or more thermal images within a thermal imaging application executing on the processor; and processing the acquired one or more thermal images in accordance with one or more processing instructions, wherein the one or more processing instructions generates one or more processed thermal image files; transmitting each of the one or more processed thermal image files of the owner; and making a determination as to whether a pandemic situation exists within a specific geographical area based on the recorded internal body temperature of the owner as stored in the one or more processed thermal data image files of the owner.

According to the first aspect of the embodiments, the at least one infrared camera device is an internal infrared camera device that is substantially completely contained within a body of the thermal data image acquisition device.

According to the first aspect of the embodiments, the at least one infrared camera device is an external infrared camera device that is electrically connected to the thermal data image acquisition device through a communications port.

According to the first aspect of the embodiments, the thermal image data acquisition device further comprises: a health monitor adapted to be worn by a non-infant person, monitor one or more vital signs of the non-infant person, generate one or more vital signs information of a non-infant person, and which is further adapted to transmit the one or more vital sign information to the thermal data acquisition device, and wherein the one or more vital signs information provides information about a health state of the non-infant person.

According to the first aspect of the embodiments, the one or more vital signs can include a heart rate, a pulse oximeter reading, and a blood pressure.

According to the first aspect of the embodiments, the step of processing the acquired one or more thermal images by the method that is executed by the processor further comprises: applying a privacy setting level to the thermal image file of the owner.

According to the first aspect of the embodiments, the privacy setting level comprises: a set of rules established by the owner of the thermal image data acquisition device that determines what types of personal data of the owner can be stored with the acquired thermal image data file, and wherein the set of rules is stored in the thermal image data acquisition device and accessed by the thermal imaging application when processing the acquired one or more thermal images of the owner, and wherein processing occurs in accordance with the set of rules established by the owner.

According to the first aspect of the embodiments, the set of rules established by the owner of the thermal image data acquisition device can be ignored by the thermal imaging application when directed by either or both of a government agency and health agency.

According to the first aspect of the embodiments, the step of making a determination by the method that is executed by the processor comprises: applying statistical analysis of the processed thermal image data files of a fixed, predetermined number of people that live within a first fixed radius of the owner of the thermal data image acquisition device to determine whether a first threshold number of people have elevated body temperatures.

According to a second aspect of the embodiments, a thermal image data acquisition device is provided, comprising: at least one wireless transceiver device, adapted to transmit and receive digital data; at least one infrared camera device, adapted to acquire one or more thermal images of one or more people in the vicinity of an owner of the thermal image data acquisition device, wherein each of the one or more thermal images records an internal body temperature of the one or more people; a processor communicatively coupled to the at least one wireless transceiver device and the at least one infrared camera device; and a memory operatively connected to the processor, wherein the memory stores computer-executable instructions that, when executed by the processor, causes the at least one processor to execute a method that comprises: acquiring, via the at least one infrared camera device, the one or more thermal images within a thermal imaging application executing on the processor; and processing the acquired one or more thermal images in accordance with one or more processing instructions, wherein the one or more processing instructions generates one or more processed thermal data image files; transmitting each of the one or more processed thermal image files of the one or more people; and making a determination as to whether a pandemic situation exists within a specific geographical area based on the recorded internal body temperature of the one or more people as stored in the one or more processed thermal image files of the owner.

According to the second aspect of the embodiments, the at least one infrared camera device is an internal infrared camera device that is substantially completely contained within a body of the thermal data image acquisition device.

According to the second aspect of the embodiments, the at least one infrared camera device is an external infrared camera device that is electrically connected to the thermal data image acquisition device through a communications port.

According to the second aspect of the embodiments, the thermal image data acquisition device further comprises: a health monitor adapted to be worn by a non-infant person, monitor one or more vital signs of the non-infant person, generate one or more vital signs information of a non-infant person, and which is further adapted to transmit the one or more vital sign information to the thermal data acquisition device, and wherein the one or more vital signs information provides information about a health state of the non-infant person.

According to the second aspect of the embodiments, the one or more vital signs can include a heart rate, a pulse oximeter reading, and a blood pressure.

According to the second aspect of the embodiments, the method that is executed by the processor further comprises: obtaining one or more thermal images of the owner of the thermal image data acquisition device.

According to the second aspect of the embodiments, the step of processing the acquired one or more thermal images by the method that is executed by the processor comprises: applying a privacy setting level to the one or more thermal images of the owner.

According to the second aspect of the embodiments, the privacy setting level comprises: a set of rules established by the owner of the thermal image data acquisition device that determines what types of personal data of the owner can be stored with the acquired thermal image data, and wherein the set of rules is stored in the thermal image data acquisition device and accessed by the thermal imaging application when processing the acquired one or more thermal images of the owner, and wherein processing occurs in accordance with the set of rules established by the owner.

According to the second aspect of the embodiments, the set of rules established by the owner of the thermal image data acquisition device can be ignored by the thermal imaging application when directed by either or both of a government agency and health agency.

According to the second aspect of the embodiments, the step of making a determination by the method that is executed by the processor comprises: applying statistical analysis of the processed thermal image data files of a fixed, predetermined number of people that live within a first fixed radius of the owner of the thermal data image acquisition device to determine whether a first threshold number of people have elevated body temperatures.

According to a third aspect of the embodiments, a thermal image data acquisition system is provided, comprising: one or more thermal image data acquisition devices, wherein each of the thermal data acquisition device comprises: at least one wireless transceiver device, adapted to transmit and receive digital data; at least one infrared camera device, adapted to acquire one or more thermal images of an owner of the thermal image data acquisition device, wherein each of the one or more thermal images records an internal body temperature of the owner; a processor communicatively coupled to the at least one wireless transceiver device and the at least one infrared camera device; and a memory operatively connected to the processor, wherein the memory stores computer-executable instructions that, when executed by the processor, causes the at least one processor to execute a method that comprises: acquiring, via the at least one infrared camera device, the one or more thermal images within a thermal imaging application executing on the processor; and processing the acquired one or more thermal images in accordance with one or more processing instructions, wherein the one or more processing instructions generates one or more processed thermal data image files; and transmitting each of the one or more processed thermal data image files of the owner; one or more thermal image data collection servers, wherein each of the one or more thermal image data collection servers comprises: at least one communications transceiver device, adapted to transmit and receive digital data; at least one second processor communicatively coupled to the at least one communications transceiver device; and a second memory operatively connected to the at least one second processor, wherein the second memory stores computer-executable instructions that, when executed by the at least one second processor, causes the at least one second processor to execute a method that comprises: receiving, via the at least one communications transceiver device, the transmitted one or more processed thermal data images files within a second thermal imaging application executing on the at least one second processor; and processing the received one or more thermal data image files in accordance with one or more processing instructions, wherein the one or more processing instructions generates one or more first aggregated/processed thermal image files; and transmitting each of the one or more first aggregated/processed thermal image data files; one or more agency thermal image data collection servers, wherein each of the one or more agency thermal image data collection servers comprises: at least one communications transceiver device, adapted to transmit and receive digital data; at least one third processor communicatively coupled to the at least one communications transceiver device; and a third memory operatively connected to the at least one third processor, wherein the third memory stores computer-executable instructions that, when executed by the at least one third processor, causes the at least one third processor to execute a method that comprises: receiving, via the at least one communications transceiver device, the transmitted one or more first aggregated/processed thermal data images files within a third thermal imaging application executing on the at least one third processor; and processing the received one or more first aggregated/processed thermal data image files in accordance with one or more processing instructions, wherein the one or more processing instructions generates one or more second aggregated/processed thermal image files; and making a determination as to whether a pandemic situation exists within a specific geographical area based on the recorded internal body temperature of the owner as stored in the one or more processed thermal data image files of the owner.

### BRIEF DESCRIPTION OF THE DRAWINGS

The above and other objects and features of the embodiments will become apparent and more readily appreciated from the following description of the embodiments with reference to the following figures. Different aspects of the embodiments are illustrated in reference figures of the drawings. It is intended that the embodiments and figures disclosed herein are to be considered to be illustrative rather than limiting. The components in the drawings are not necessarily drawn to scale, emphasis instead being placed upon clearly illustrating the principles of the aspects of the embodiments. In the drawings, like reference numerals designate corresponding parts throughout the several views.

FIG. 1 illustrates a conceptual block diagram of a thermal scanning system for substantially automatically thermally scanning an owner of a mobile electronic device that is part of the thermal scanning system according to aspects of the embodiments.

FIG. 2 illustrates a flow chart of a method for operating the thermal scanning system shown in FIG. 1 according to aspects of the embodiments.

FIG. 3 illustrates a block diagram of a thermal data collection and aggregation network that can be used to facilitate thermal data collection, aggregation, and analysis according to aspects of the embodiments.

FIG. 4 illustrates a flow chart of a method for performing thermal image data analytics according to aspects of the embodiments.

FIG. 5 illustrates a block diagram of the major components of a computer device, personal computer, server, laptop, and/or personal electronic device (herein after, “computer device”) suitable for use to implement one or more of the methods shown in regard to FIG. 2 or 4, as well being a component of the network shown in FIG. 3 according to aspects of the embodiments.

FIG. 6 illustrates a block diagram of a network system within which the thermal scanning system of FIG. 1 can be implemented according to aspects of the embodiments.

### DETAILED DESCRIPTION

The embodiments are described more fully hereinafter with reference to the accompanying drawings, in which embodiments of the inventive concept are shown. In the drawings, the size and relative sizes of layers and regions may be exaggerated for clarity. Like numbers refer to like elements throughout. The embodiments may, however, be embodied in many different forms and should not be construed as limited to the embodiments set forth herein. Rather, these embodiments are provided so that this disclosure will be thorough and complete, and will fully convey the scope of the inventive concept to those skilled in the art. The scope of the embodiments is therefore defined by the appended claims.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” or “in an embodiment” in various places throughout the specification is not necessarily referring to the same embodiment. Further, the particular feature, structures, or characteristics may be combined in any suitable manner in one or more embodiments.

### LIST OF REFERENCE NUMBERS FOR THE ELEMENTS IN THE DRAWINGS IN NUMERICAL ORDER

The following is a list of the major elements in the drawings in numerical order.
100 Thermal Scanning System (TSS)
102 Mobile Electronic Device (MED)
104 Processor
106 Memory
107 Thermal Image Data
108 Thermal Imaging Application (TI App)
109 Thermal Image Data File
110 Internal Communications/Data Bus (Bus)
111 Processed Thermal Image Data File
112 Cellular Wireless Transceiver
114 Bluetooth Transceiver
116 WiFi Transceiver
117 Satellite Transceiver
118 Input/Output (I/O) Port
120 Touch-Screen
122 Speaker
124 Microphone (Mic)
126 Internal Camera
128 Lens
130 Visible Spectrum Sensor Array
132 Infrared (IR) Spectrum Sensor Array
134 External Camera
136 Infant Health Monitor
138 Adult Health Monitor
200 Method for Use of Thermal Scanning Device 100
202-208 Method Steps of Method 200
300 Thermal Image Data Collection and Aggregation Network
302 Communications Network
304 Cellular Communications Network
306 Internet (and Other Electronic Communications Networks)
308 Thermal Image Data and Related Information Aggregator Network Server (TI Data Server)
310 Local Thermal Image Data Information Analytics Application (Local TI App)
311 Database Server (Database)
312 Network Server for One or More Government/Health Organizations (e.g., CDC, WHO, Local Health Department, State Health Department, Among others; Hereinafter referred to as “Agency Server”)
314 Master Thermal Image Data and Related Information Analytics Application (Master TI App)
315 First Aggregated/Processed Thermal Image Data
317 Second Aggregated/Processed Thermal Image Data
400 Method for Performing Thermal Image Data Analytics
402-410 Method Steps of Method 400
501 Shell/Box
502 Integrated Display
504 Internal Data/Command Bus (Bus)
506 Processor(s)
508 Memory
510 Universal Serial Bus (USB) Port
511 Ethernet Port
512 Compact Disk (CD)/Digital Versatile Disk (DVD) Read/Write (RW)
514 Floppy Diskette Drive
516 Hard Disk Drive (HDD)
518 Read-Only Memory (ROM)
520 Random Access Memory (RAM)
522 Video Graphics Array (VGA) Port or High Definition Multimedia Interface
524 External Memory Storage Device
526 External Display
528 Keyboard
530 Mouse
532 Internal Memory
534 Flash Drive Memory
536 CD/DVD Diskettes
538 Floppy Diskettes
542 Wi-Fi Transceiver
544 BlueTooth (BT) Transceiver
546 Near Field Communications (NFC) Transceiver
548 Third Generation (3G), Fourth Generation (4G), Long Term Evolution (LTE),
550 Communications Satellite/Global Positioning System (Satellite) Transceiver
552 Antenna
556 Universal Serial Bus (USB) Cable
558 Ethernet Cable (CATS Cable)
560 Printer
562 VGA/HDMI Cable
600 Network
606 Internet Service Provider (ISP)
608 Modulator/Demodulator (Modem)
610 Wireless Router
612 Plain Old Telephone Service (POTS) Provider
614 Cellular Service Provider
618 Communication Satellites
620 Cellular Telecommunications Service Tower (Cellular Tower)
624 GPS Station
626 Satellite Communication Systems Control Station
628 Global Positioning System (GPS) Satellite

### LIST OF ACRONYMS USED IN THE SPECIFICATION IN ALPHABETICAL ORDER

The following is a list of the acronyms used in the specification in alphabetical order.
3G Third Generation
4G Fourth Generation
5G Fifth Generation
API Application Programming Interface
App Executable Software Programming Code/Application
ASIC Application Specific Integrated Circuit
BIOS Basic Input/Output System
BT BlueTooth
CD Compact Disk
CDC Center for Disease Control
CRT Cathode Ray Tube
DVD Digital Video Disk
EEPROM Electrically Erasable Programmable Read Only Memory
FPGA Field Programmable Gate Array
GAN Global Area Network
GDPR General Data Protection Regulation
GPS Global Positioning System
GUI Graphical User Interface
HDD Hard Disk Drive
HDMI High Definition Multimedia Interface
IR Infra-red
ISP Internet Service Provider
LCD Liquid Crystal Display
LED Light Emitting Diode Display
LTE Long Term Evolution
MED Mobile Electronic Device
Mic Microphone
MODEM Modulator-Demodulator
NFC Near Field Communication
NW Network
PC Personal Computer
PDA Personal Digital Assistant
PDF Portable Document Form
PED Personal Electronic Device
PFF People Finder Feature
POTS Plain Old Telephone Service
PROM Programmable Read Only Memory
PSL Privacy Setting Level
RAM Random Access Memory
ROM Read-Only Memory
RW Read/Write
TI Thermal Image
TI Thermal Imaging
TSI Thermal Scanning Information
TSS Thermal Scanning System
UN United Nations
URL Uniform Resource Locator
USB Universal Serial Bus
UV Ultraviolet Light
WHO World Health Organization

Aspects of the embodiments, as described below in reference to the Figures provided herein, provide at least the functions described herein to overcome the problems in detecting and tracking infective outbreaks, and notifying the people so affected and government and health agencies of such infective outbreaks.

The different aspects of the embodiments described herein pertain to the context of systems, methods, and modes for a personal mobile electronic communications device that can automatically record a thermal image of a user-owner of the personal mobile electronic communications device, as well as to thermally image others by the same personal mobile electronic communications device, but is not limited thereto, except as may be set forth expressly in the appended claims.

As briefly described above, aspects of the embodiments are directed to systems, methods, and modes for a personal mobile electronic communications device that can automatically record a thermal image of a user-owner of the personal mobile electronic communications device, as well as to thermally image others by the same personal mobile electronic communications device. The ownership of personal mobile electronic devices (herein after “mobile electronic device” or “MED”) 102 is substantially ubiquitous in the United States and many other industrialized nations, and beginning to become prevalent in developing countries as well. Such MED 102 can include cellular phones, tablets, iPads, and the like, personal digital assistants (PDAs), among other personal communication type devices.

In the following detailed description, references are made to the accompanying drawings that form a part hereof, and in which are shown by way of illustrations, specific embodiments, or examples. These aspects may be combined, other aspects may be utilized, and structural changes may be made without departing from the spirit or scope of the present disclosure. The following detailed description is therefore not to be taken in a limiting sense, and the scope of the present invention is defined by the appended claims and their equivalents.

While some embodiments will be described in the general context of program modules that execute in conjunction with an application program that runs on an operating system on a MED 102, personal computer and/or specifically designed and fabricated electronic devices, those skilled in the art will recognize that aspects may also be implemented in combination with other program modules.

Generally, program modules include routines, programs, components, data structures, and other types of structures that perform particular tasks or implement particular abstract data types. Moreover, those of skill in the art can appreciate that different aspects of the embodiments can be practiced with other computer system configurations, including hand-held devices, multiprocessor systems, microprocessor-based or programmable consumer electronics, minicomputers, mainframe computers, and comparable computing devices. Aspects of the embodiments can also be practiced in distributed computing environments where tasks are performed by remote processing devices that are linked through a communications network. In a distributed computing environment, program modules can be located in both local and remote memory storage devices.

Aspects of the embodiments can be implemented as a computer-implemented process (method), a computing system, or as an article of manufacture, such as a computer program product or computer readable media. The computer program product can be a computer storage medium readable by a computer system (e.g., such as one used on MED 102) and encoding a computer program that comprises instructions for causing a computer or computing system to perform example process(es). The computer-readable storage medium is a computer-readable memory device. The computer-readable storage medium can for example be implemented via one or more of a volatile computer memory, a non-volatile memory, a hard drive, a flash drive, a floppy disk, or a compact disk, and comparable hardware media. Other hardware components can include one or more of externally attachable thermal scanners or infra-red cameras.

Throughout this specification, the term “platform” can be a combination of software and hardware components for providing share permissions and organization of content in an application with multiple levels of organizational hierarchy. Examples of platforms include, but are not limited to, a hosted service executed over a plurality of servers, an application executed on a single computing device, and comparable systems. The term “server” generally refers to a computing device executing one or more software programs typically in a networked environment. More detail on these technologies and example operations is provided below.

A computing device, as used herein, refers to a device comprising at least a memory and one or more processors that includes a server, a desktop computer, a laptop computer, a tablet computer, a smart phone, a vehicle mount computer, or a wearable computer. A memory can be a removable or non-removable component of a computing device configured to store one or more instructions to be executed by one or more processors. A processor can be a component of a computing device coupled to a memory and configured to execute programs in conjunction with instructions stored by the memory. Actions or operations described herein may be executed on a single processor, on multiple processors (in a single machine or distributed over multiple machines), or on one or more cores of a multi-core processor. An operating system is a system configured to manage hardware and software components of a computing device that provides common services and applications. An integrated module is a component of an application or service that is integrated within the application or service such that the application or service is configured to execute the component. A computer-readable memory device is a physical computer-readable storage medium implemented via one or more of a volatile computer memory, a non-volatile memory, a hard drive, a flash drive, a floppy disk, or a compact disk, and comparable hardware media that includes instructions thereon to automatically save content to a location. A user experience can be embodied as a visual display associated with an application or service through which a user interacts with the application or service. A user action refers to an interaction between a user and a user experience of an application or a user experience provided by a service that includes one of touch input, gesture input, voice command, eye tracking, gyroscopic input, pen input, mouse input, and keyboards input. An application programming interface (API) can be a set of routines, protocols, and tools for an application or service that allow the application or service to interact or communicate with one or more other applications and services managed by separate entities.

Aspects of the embodiments address a need that arises from pandemics that can cause sickness and death, and use an incredible amount of resources, such as money, medical supplies, the time and expertise of physicians and nurses and others in the healthcare industry, as well as the time, energy, and effort of government officials. As has been witnessed by billions of people, and as discussed above, the recent novel coronavirus pandemic has caused well over 300,000 deaths globally, over a million infections, the collapse of the global economy, the loss of innumerable jobs, and cost billions of dollars as well. The actions/operations described herein are not a mere use of a computer, but address results of a system that is a direct consequence of software used as a service such as communication services offered in conjunction with communications.

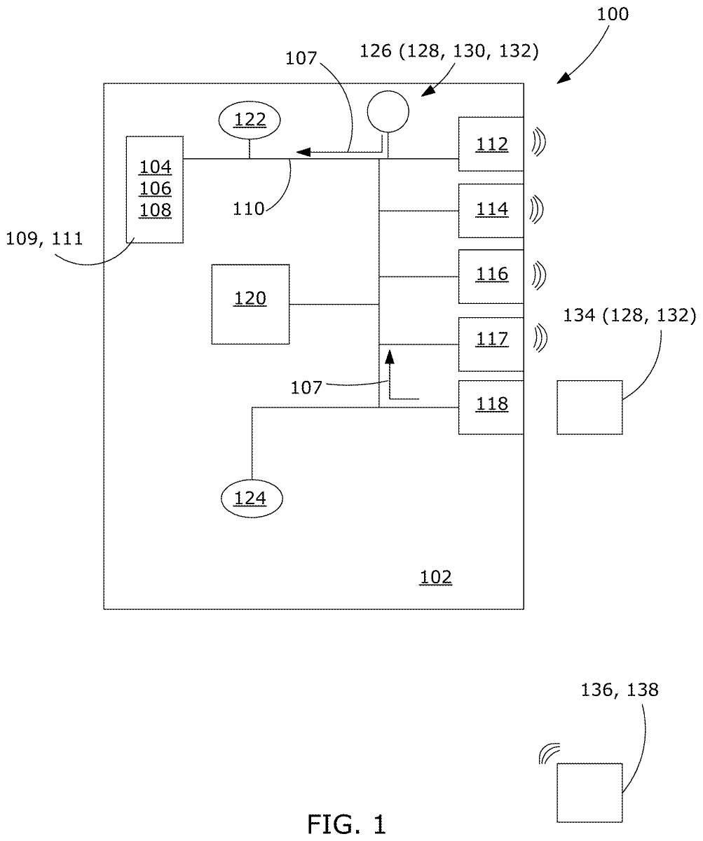

FIGS. 1-6 illustrate various aspects of a computer and network-based personal thermal data collection and aggregating system, including, according certain aspects of the embodiments, use of the internet or other similar networks. The personal thermal data collection and aggregating system personal thermal data collection and aggregating programs provides a practical, technical solution to the problem of collecting, processing aggregating and distributing personal thermal information to medical and governmental officials; as those of skill in the art can appreciate, the aspects of the embodiments has no “analog equivalent” as its embodiments reside solely or substantially in the physical device or computer domain. That is, managing the inventory of personal thermal data collection and aggregating system that can be stored and acquired over vast areas, as well as integrating the thermal information with other information, much of which can only be obtained electronically, means using practical, non-abstract physical devices. The technological improvement of the aspects of the embodiments resides in at least in the ability to quickly and easily integrate personal thermal information from one or more people in any given area, and integrate that with personal thermal information from a large number of people from numerous and disparate areas, wherein all of the data can only be obtained electronically in any practical or real sense according to aspects of the embodiments.

In addition, such aspects of the embodiments have no “analog equivalents” because the various aspects of the embodiments' algorithm not only can acquire personal thermal information from hundreds of thousands, if not millions of people from a distance and automatically, but can combine the electronically obtained thermal information with relevant population statistical information (housing density, economic indicators, among other related information) with environmental information (a dry, cold environment might present a greater risk of infected people infecting others, versus humid, high temperature environments), that is, in many cases, can only be practically obtained and combined through electronic means, and allows users to obtain and search the combined personal aggregated thermal information using many different types of commands none of which could be accomplished without a computer or some other technological equivalent.

FIG. 1 illustrates a conceptual block diagram of thermal scanning system (TSS) 100 for substantially automatically thermally scanning an owner of mobile electronic device (MED) 102 that is part of thermal scanning system 100 according to aspects of the embodiments.

As discussed above, many people in industrialized nations such as the United States, Canada, Japan, all of Europe, and many others, have at least one “personal mobile electronic communications device,” (MED) 102, or cell phone. Further, many of these are so-called “smart” phones incorporate at least two or more cameras therein: a camera on the rear surface to take photos of objects facing the user, and one in the front, to take photos of the user, or “selfies.” According to aspects of the embodiments the MED 102 of TSS 100 includes also at least one infrared camera that can capture thermal images of a user/owner of MED 102. According to further aspects of the embodiments, a first example of MED 102 includes a built-in thermal or infrared camera, and a second example of MED 102 includes an attachable thermal or infrared camera.

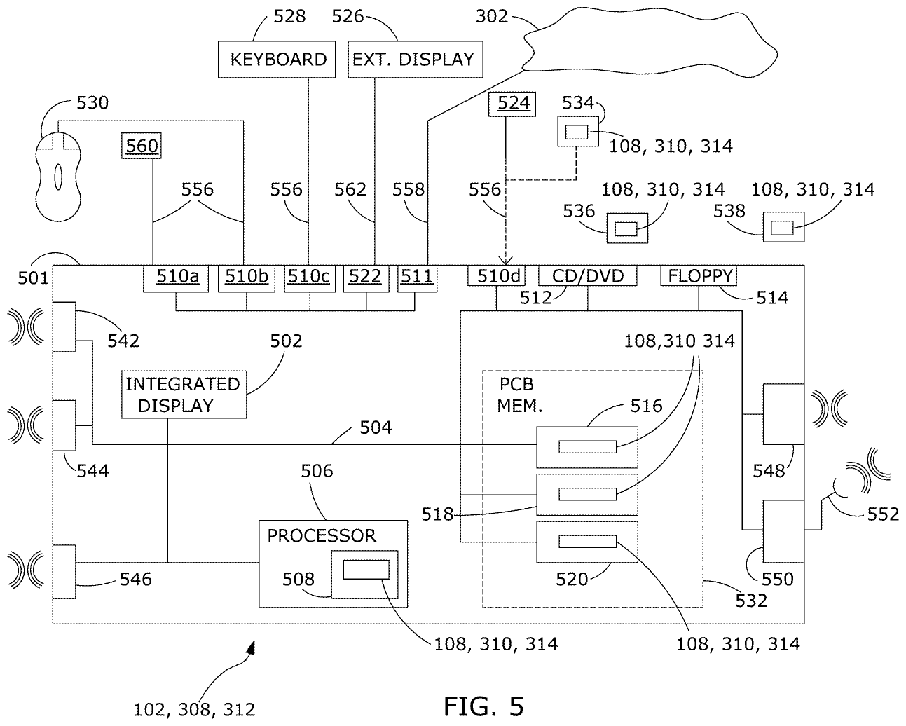

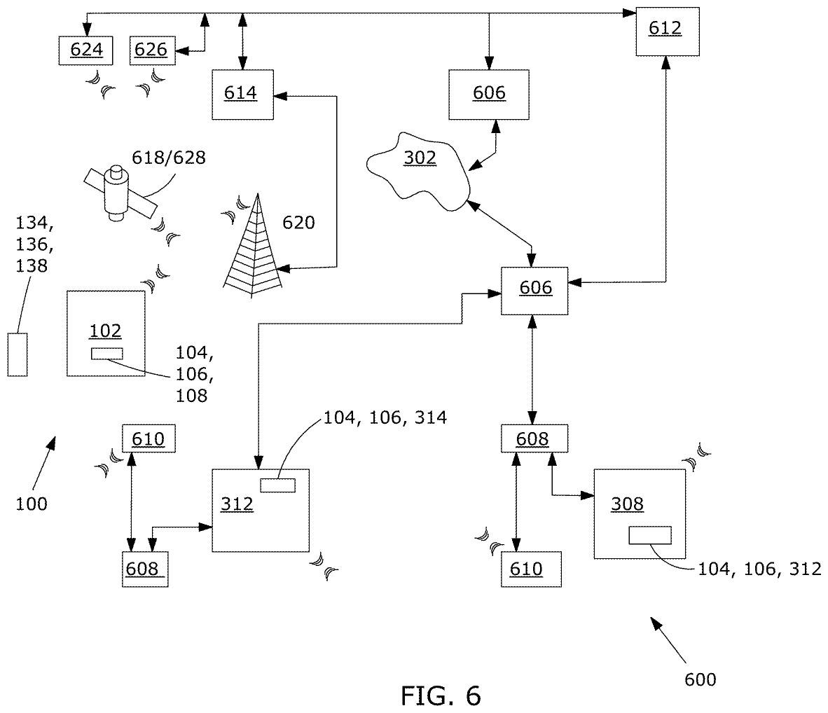

Referring to FIG. 1, MED 102 comprises processor 104, memory 106, and thermal imaging application (TI App) 108, which is stored in either a memory 106 that is part of processor 104, or which is accessible to processor 104 via bus 110. MED 102 further comprises one or more of cellular wireless transceiver 112 (to communicate wirelessly with one or more cellphone towers (shown and discussed below in regard to FIG. 6) as part of a wireless cellular telecommunication's system), Bluetooth transceiver 114 (discussed in greater detail below in regard to FIGS. 5 and/or 6), WiFi transceiver 116 (also discussed in greater detail below in regard to FIGS. 5 and/or 6), and satellite transceiver 117 (also discussed in greater detail below in regard to FIGS. 5 and/or 6). Each of the transceivers 112, 114, 116 have their own antennas and receiving/transmitting portions that have been omitted in the Figures in fulfillment of the dual purposes of clarity and brevity; those of skill in the art appreciate their operation and use in MED 102. Transceivers 112, 114, and 116 provide means for communicating to different devices in different manners to accomplish different tasks. Although a detailed discussion of each is both beyond the scope of this discussion, and not needed by those of skill in the art to appreciate the various aspects of the embodiments described herein and in the one or more claims, a brief review is useful.

Cellular wireless transceiver 112 allows the use of MED 102 in a wireless cellular telephone network, providing telephonic audio and data communications virtually anywhere provided there is “coverage” by one or more cell phone towers of the wireless carrier the user subscribes to. “Coverage” refers to transmission and reception of electromagnetic signals that are sufficient to allow for the electromagnetic transfer of audio and data signals. Bluetooth transceiver 114 is substantially similar to that of cellular wireless transceiver 112, but, in general has a much shorter transmission range and is defined by substantially different transmission/reception protocols. Many devices that employ Bluetooth transceivers utilize them for the purposes of wireless headsets, speakers, and other devices that generally are used solely by the owner/operator of MED 102 or a relatively few amount of other people. For example, exercise heartrate monitors use Bluetooth in many cases. A software program, or communications Application (App) facilitates communications to and from the remote device through MED 102. Such operations and functionality are well known to those of skill in the art. WiFi transceiver 116 also allows for wireless transmission/reception of audio/data, but has a much greater range than that of Bluetooth, but much less than that of cellular wireless transceiver 112. Many users make use of WiFi transceiver 116 and its functionality to operate devices remotely within their homes, and to communicate with their wireless cell phone service provider (through their internet service provider) for calls/data transmissions in order to not use cell phone tower transmissions, as those can incur costs for excessive use of data. According to aspects of the embodiments, any one or more of transceivers 112, 114, 116can communicate the acquired personal thermal data in the manner to be described in greater detail below. MED 102 further comprises one or speakers 122 and microphones (mic) 124.

According to further aspects of the embodiments, MED 102 further comprises one or more internal cameras 126, and external camera 134. Internal camera 126 comprises one or more lenses 128, one or more visible light spectrum sensor arrays 130, and one or more infrared (IR) sensor arrays 132 according to aspects of the embodiments, MED 102 can also include a forward looking lens 128 (i.e., on the side opposite to the one that contains the screen 120, and speaker(s) 122 and mic(s) 124). In addition to internal camera 126, TSS 100 further comprises external camera 134 according to aspects of the embodiments.

According to an aspect of the embodiments, camera 126 comprises one lens 128, one visible spectrum sensor array 130, and an infrared spectrum sensor array 132. As those of skill in the art can appreciate, rearward facing sensor arrays are typically smaller and have fewer photoreceptors that make up the sensor array than forward facing cameras, which may even have up to three lenses 128. When taking photos of an object, internal or external camera 126, 134 can capture the light coming from it. Convex lens 128 can be used to focus incoming light onto one or more of visible spectrum sensor array 130 and IR spectrum sensor array 132. The sensors 130, 132 can then create a digital photograph from the incoming light.

According to aspects of the embodiments, while lenses 128 can be designed and fabricated to pass substantially only visible light wavelengths, it can be the case that lens 128 has a sufficiently wide enough bandwidth to allow both visible light and infrared light onto respective sensor arrays 130, 132. According to aspects of the embodiments, such photoreceptor sensor arrays 130, 132 can be wavelength sensitive such that a wavelength other than that which they were designed to receive and respond to would be ignored. Or, in other words, if visible light wavelengths were to fall on IR spectrum sensor array 132, that light would be substantially ignored, and visa-versa with IR light that falls upon visible spectrum sensor array 130. According to further aspects of the embodiments, one or more mirrors in conjunction with wavelength filters can be used to substantially eliminate undesired wavelengths of light from being received by the sensor arrays. That is, according to one non-limiting aspect of the embodiments, light can be received by lens 128 and is then split into two light beams. A first light beam, containing both IR and visible light wavelength components, is filtered by an IR blocking filter, and then the filtered first light beam is received by visible spectrum sensor array 130, wherein substantially all of the IR light has been blocked before the light enters visible spectrum array 130. A second light beam (substantially similar to first light beam after being split by the mirror), is filtered by a visible light blocking filter prior to being sent to IR spectrum sensor array 132. The filtered second light beam is received by IR spectrum sensor array 132, wherein substantially all of the visible light has been blocked before the light enters IR spectrum array 132. These optical arrangements, which have been greatly simplified in this discussion in fulfillment of the dual purposes of clarity and brevity, are known to those of skill in the art. The aspects of the embodiments pertain to the specific use of IR spectrum sensor arrays 132 in conjunction with the thermal image reporting applications and thermal data aggregation application as discussed below.

External camera 134 works substantially similarly to that of internal camera 126; however, it can be the case that external camera 134 has only IR spectrum sensor array 132, and such has been shown in FIG. 1 according to aspects of the embodiments. External camera 134 can either have a short, substantially nonflexible connector that can be attached to IO port 118, or can have a short/long cord that can have a mating connector to IP port 118 according to aspects of the embodiments.

According to aspects of the embodiments, as shown in FIG. 1, the output of IR spectrum sensor array is thermal image data 107. Thermal image data 107 is the “raw” output of the camera prior to storing and/or further processing “upstream” in MED 102 and other components of the aspects of the embodiments. Although it could be the case that the outputs of internal camera 126 and external camera 134 (in regard to the IR spectrum) is formatted differently, or has different resolution, among other differences, for the purposes of this discussion, in fulfillment of the dual purposes of clarity and brevity, the outputs of internal and external cameras 126, 134 will be considered to be substantially similar and denoted as thermal image data 107.

According to further aspects of the embodiments, external camera 134 can be used in both a rearward and forward-facing direction. In the case in which external camera 134 can be used in both rearward (i.e., the “selfie” mode) and forward-facing direction, there can be two cameras—i.e., two separate lenses 128 and two separate IR spectrum sensor array 132. According to further aspects of the embodiments, a single IR spectrum sensor array 132 can be paired with two different lenses 128, with the use of mirrors to redirect the light to IR spectrum sensor array 132. As those of skill in the art can appreciate, this discussion has been greatly simplified in fulfillment of the dual purposes of clarity and brevity and other optical configurations are possible.

According to aspects of the embodiments, optional devices that can be associated with TSS 100 can include one or more of an infant health monitor 136 and an adult health monitor 138. Each of devices 136, 138 can communicate with MED 102 using Bluetooth and/or WiFi communication protocols. Each of devices 136, 138 monitor one or more of temperature, pulse/heart rate, blood oxygen level, and can even monitor blood pressure according to aspects of the embodiments. Infant health monitor 136 can be used on infants to small children, and adult health monitor can be used for small children to adults. TI App 108 can incorporate questions about additional family members and monitoring of their “vital signs” in regard to the various aspects of the embodiments. According to aspects of the embodiments, at times of a pandemic, or at any time, one or more of the government and/or health agency can require one or more of a population to wear devices 136, 138 in order to track and monitor the health of the population. Such devices 138 can be used at schools such as colleges, or other private institutions, including hospitals and other medical care facilities.

According to further aspects of the embodiments, MED 102 further comprises thermal imaging application (TI App) 108, which can be stored memory 106 that can be either part of processor 104, external to processor 104, or some combination thereof.

According to aspects of the embodiments, TI App 108 can be pre-installed in every MED 102 sold in a particular country or region, and its use can be mandatory.

According to aspects of the embodiments, TI App 108 can be pre-installed in every MED 102 sold in a particular country or region, and its use can be voluntary.

According to aspects of the embodiments, TI App 108 can be pre-installed in every MED 102 sold in a particular country or region, and its use can be voluntary, unless a government authority decides that its use must be mandatory, and it issues a directive to all cellular phone service providers to enable automatic use of TI App 108.

According to aspects of the embodiments, the use TI App 108 is such that thermal image data 107 of the owner/operator of MED 102 is acquired one or more times a day, typically in the morning, and again during the day, and again at night. Such thermal image data 107 can be acquired according to a pre-arranged, pre-determined schedule, or they can be set up to be completely random, or some combination thereof. The thermal image data 107 can be acquired randomly on some days, and periodically on others. The days in which thermal image data 107 acquisition occurs randomly can itself be randomized, as well as the day in which thermal image data 107 is acquired according to a known, set schedule (i.e., periodically) can also be randomized. Should MED 102 also include a forward-facing internal camera 126, as most MEDs 102 do, TI App 108 can further acquire thermal image data 107 of others in any of the manners described above according to aspects of the embodiments.

According to aspects of the embodiments, thermal image data 107 is received by processor 104, whether internally or externally generated, and stored as thermal image data file 109. Once TI App 108 processes thermal image data file 109, it becomes processed thermal image data file 111. Processed thermal image data file 111 is described in greater detail below, and is the thermal data of the owner of the MED 102 or others that is eventually transmitted to other components of TSS 100, and thermal image data collection and aggregation network

According to aspects of the embodiments, TI App 108, through operation of

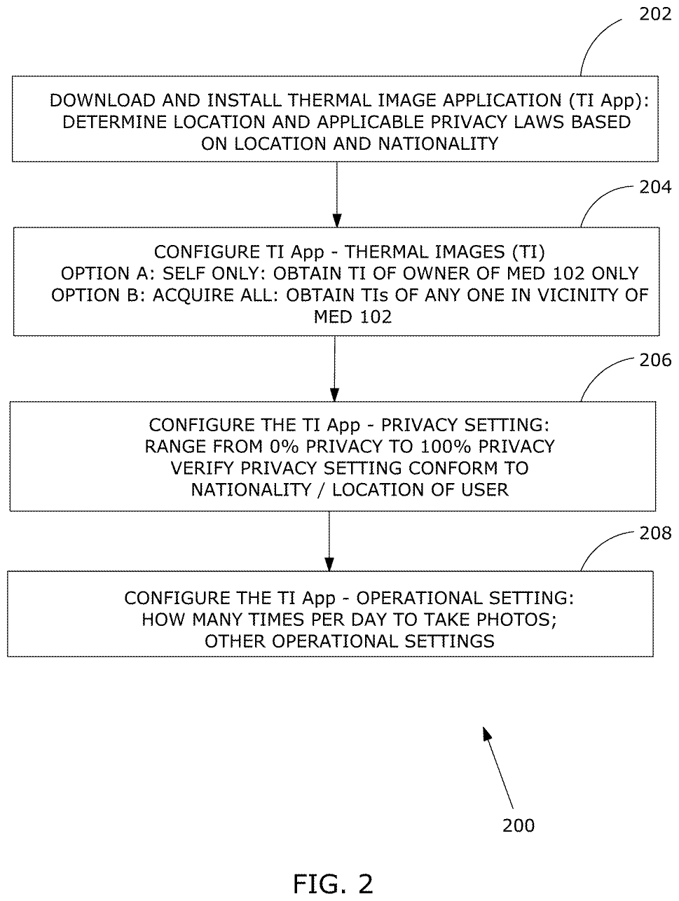

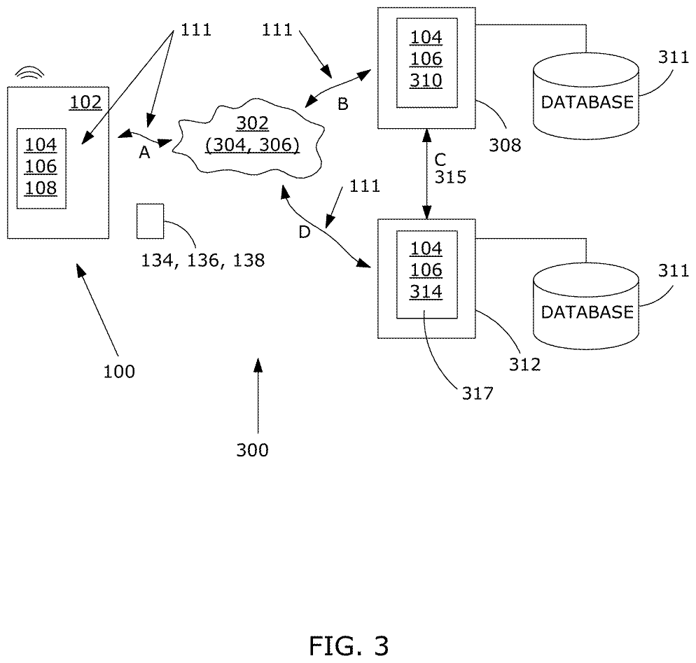

MED 102, can access one or more networks to distribute processed thermal image data file 111 of the owner/operator of MED 102 among others, in a manner to be described in greater detail below. Attention is now directed to FIGS. 2 and 3.

FIG. 2 illustrates a flow chart of method 200 for operating TSS 100 shown in FIG. 1 according to aspects of the embodiments, and FIG. 3 illustrates a block diagram of a thermal data collection and aggregating network that can be used to facilitate thermal data collection, aggregation, and analysis according to aspects of the embodiments

Method 200 begins with method step 202 in which a user of MED 102 acquires MED 102 and TI App 108 is either pre-installed or can be downloaded in a manner known to those of skill in the art. According to aspects of the embodiments, installation of TI App 108 can be mandated by the government and done at the time of purchase.

According to further aspects of the embodiments, TI App 108, once downloaded, performs several functions.

One of the first functions TI App 108 performs is to ascertain the geographical location of MED 102, with or without permission of the owner, and review applicable privacy laws that can be stored in database 311, as shown in FIG. 3. TI App 108 performs this function almost instantaneously to determine what personal information can and cannot be collected legally. In some states and countries, the amount, type, and durations of personal information that can be stored is strictly limited. See, e.g., The General Data Protection Regulation (GPDR), at https://ec.europa.eu/info/law/law-topic/data-protection/data-protection-eu_en). Certain states in the United States have also enacted personal data protection laws; See, e.g., https://oag.ca.gov/privacy/privacy-laws; other states and the Federal Government have also enacted privacy laws as well. TI App 108 determines the geographical location of MED 102.

According to further aspects of the embodiments, TI App 108 also contains the uniform resource location (URL) addresses of one or more of servers 308 to forward acquired thermal image data (the URL is also commonly referred to as the webpage address). Such transmissions of acquired thermal image data will be discussed in greater detail below in regard to FIG. 3, and thermal data collection and aggregation network. According to aspects of the embodiments, TI App 108 verifies that MED 102 can communicates with one or more servers 308 as soon as possible upon startup, initialization, and enablement of MED 102.

According to aspects of the embodiments, there can be a server 308 for every state, or a plurality of servers 308 in each state, or multiple servers for larger population areas such as large cities (e.g., New York, Chicago, Los Angelos, among others).

Method 200 then proceeds to method step 204 and begins the process of configuring the TI App 108 for use in the particular MED 102 it has been downloaded and/or enabled in, and according to the privacy laws as determined in method step 202. According to aspects of the embodiments, it can be presumed that no privacy laws exist, or that privacy laws can be overridden in the event of a national health emergency. According to further aspects of the embodiments, TI App 108 can allow for a person to opt-in or opt-out of protection by relevant privacy laws. In such cases of true emergencies, and/or opt-out, any and all personal information can be collected as needed in order to provide the greatest amount of medically relevant information needed to ascertain the scope, severity, and growth of a pandemic illness. According to further aspects of the embodiments, a determination by a globally recognized health organization or a federal or a local government agency that a pandemic illness is present in a specific geographical area, or likely to be present, can override any and all privacy laws and concerns.

In method step 204, TI App 108 collects basic personal information including, but not limited to one or more of name, phone number(s), email addresses, age, gender, home and work address(es), family size, co-habitants, weight, generally healthiness, the presence or absence of certain illnesses or conditions (so called “comorbidity” factors, according to specific pandemic illness, e.g., diabetes, high blood pressure, asthma, among others), among other information. TI App 108 then causes MED 102 to obtain a photo of the face of the owner of MED 102, in both the regular visible spectrum and thermal image data 107, using IR spectrum sensor array 132, in the manner as described above.

According to aspects of the embodiments, the owner of MED 102 further determines whether TI App 108 acquires thermal image data 107 of just the owner (operating in “Acquire Self-Only” mode) or is operated in a public mode setting (“Acquire ALL”). That is, if TI App 108 is operated in the self-mode setting, only thermal image data 107 of the owner is acquired, in a manner described in greater detail below. If TI App 108 were to be operated in the “Acquire ALL” mode setting, then TI App 108 would acquire thermal image data 107 of practically anyone in a manner to be described in greater detail below.

Following method step 204, method 200 proceeds to method step 206. In method step 206, the user configures a privacy setting level (PSL) in TI App 108, according to further aspects of the embodiments.

The PSL can range from 1-10, or 0-100%, or some other such scale, in order to indicate maximum privacy (10, or 100%), or no privacy whatsoever (0, or 0%). According to aspects of the embodiments, an owner of MED 102 may decide, even in the absence of an overriding public health emergency, that he or she wants to provide as much health related information to the agency or agencies that collect data from a plurality of MEDs 102 and thermal scanning information network (TSI NW) 300—which would indicate a privacy setting of 0 or 0%. In such case, any and all health-related information can be sent through TSI NW 300 and stored in memory 106 of server 308 and/or database server 311 that is associated with server 308. If the owner of MED 102 decides to maximize the PSL, then TI App 108 may only send processed thermal image data file 111 of the owner and relative location, such as a zip code, or a more specific location, such as a cellular communications based location determination or a global position determining system (GPS) location (latitude and longitude); as those of skill in the art can appreciate, some of these position determination systems are more exact than others. According to further aspects of the embodiments, each piece of personal information that can be added to processed thermal image data file 111 decreases the PLS. By way of non-limiting example, Table 1 below illustrates but one example of a spectrum of PSLs that can be used in the aspects of the embodiments and MED 102, TSS 100, and methods 200 and 400.

TABLE 1
Privacy Setting Level

| 0% | 25% | 50% | 75% | 100% |
|---|---|---|---|---|
| 1. Thermal | 1. Thermal | 1. Thermal | 1. Thermal | 1. Thermal |
| Image | Image | Image & | Image & GPS | Image & |
| 2. Personal | 2. Personal | Relative | Location | Relative |
| Information | Information | Location | | Location |
| 3. Exact GPS | 3. Exact GPS | 2. Personal | | |
| Location | Location | Information | | |
| 4. Nearby | | | | |
| People (People | | | | |
| Finder Feature) | | | | |

According to aspects of the embodiments, Table 1 indicates only one of innumerable PSL setting scenarios, and in practice, the scale can be significantly more detailed; in fulfillment of the dual purposes of clarity and brevity, however, Table 1 illustrates the aspects of the embodiments in sufficient detail to those of skill in the art, and equivalents thereto are considered to be part of the aspects of the embodiments.

According to further aspects of the embodiments, an owner of MED 102 could be offered money as an inducement to set their PSL to 0 or 0%, and such money could be a first fixed sum, and different sums can be offered for differing levels of PSLs.

Shown in Table 1, but not previously discussed, is the “People Finder Feature” of TI App 108 according to aspects of the embodiments. The “People Finder Feature” (PFF) can be a pre-programmed pre-loaded aspect of TI App 108 that uses one or more of WiFi, Bluetooth, and/or cellular provided signaling/communications mechanisms to locate and identify people and their MEDs 102 that are nearby to the owner of the MED 102. According to aspects of the embodiments, the PFF can be autonomously/remotely activated (e.g., by the government, or duly appointed and approved local/national/international health agency), or it can be permanently activated, or it can be self-activated by the owner of MED 102. In the first two instances, the autonomously and permanently activated functionality, the PFF is substantially permanently operating and finds other MEDs 102 that also have TI App 108 and the PFF portion. In the other case, the self activated mode, the PFF would only find other MEDs 102 that have also self-activated the PFF portion of TI App 102. Those of skill in the art can appreciate that use of PFF in TI App 102 provides a manner of tracing—i.e., locating others that might have come in contact with a person that has a high body temperature that can indicate a commutable illness such as the novel coronavirus. Substantially fully automated, use of the PFF of TI App 102 can provide a very cost effective and efficient means of locating people that have or might have come in contact with a person with a communicable disease. Such a feature can save hundreds of lives and significant amounts of money. Automated messages can be sent to those determined to have been in relatively close contact to those that are or become ill or exhibit symptoms of illness. Additional safeguards can be put into place to avoid false positives, such as not messaging unless and until the original person exhibiting symptoms such as higher body temperatures is tested for a particular communicable illness, as there are many reasons for elevated body temperatures in which tracing is not appropriate. According to further aspects of the embodiments, people that do exhibit a higher than normal body temperature as exhibited by the reporting of thermal images can be sent messages and requested/required to confirm diagnosis, or health care facilities can be instructed to auto-report the results.

Following method step 206, method 200 proceeds to method step 208.

In method step 208, configuration of TI App 108 continues are in regard to operational settings. One of several different operations settings is how many times a day and at what time of the day will TI App 108 acquire thermal image data 107 of the owner of MED 102. For example, it is known by those of skill in the art that on average, an individual's core body temperature varies from 36.5-37.5° C. (97.7-99.5° F.) over the course of a day. A typical body temperature does not remain constant however, as it fluctuates according to a person's circadian rhythm; in fact, this rhythm tends to represent a sinusoidal shape over the course of the day. Typically, a person's body temperature is at its lowest first thing in the morning, and highest just before bedtime. According to aspects of the embodiments, one setting could be to acquire a thermal image of the owner of MED 102 as soon as possible after waking up in the morning (this, of course, presumes that the owner does not work late or very early morning shifts, such as 1200 AM to 0800 AM). Other settings could be to acquire thermal image data 107 of the owner of MED 102 periodically (e.g., every hour or so, if able), or at random during the course of the day, or any/all combinations thereof. According to further aspects of the embodiments, the acquisition of thermal image data 107 of the owner of MED 102 can be limited to N thermal image data 107, or not limited at all, and the same can be in regard to acquired thermal image data 107 of other people.

According to further aspects of the embodiments, an audio or sensory alert can be issued when it is time to acquire thermal image data 107 of the owner of MED 102; that is, if TI App 108 is configured to acquire at least one thermal image data 107 of the owner of MED 102 in the morning, and that has not occurred by at least 10 AM, and TI App 108 determines that MED 102 is being used or has been used, TI App 108 can request thermal image data 107 be acquired, prior to any other uses of MED 102 according to aspects of the embodiments. The alerts can be in the form of an audible announcement, whether a spoken command or noise (such as a buzzer), a light (most MEDs 102 include lamps for taking photos in low light conditions, and this can start flashing until thermal image data 107 is acquired), an automated telephone call to the device, or virtually any combination of these and other indicators according to aspects of the embodiments. Following method step 208, TI App 108 is substantially fully configured and can proceed to operate autonomously, or through direction of the owner of MED 102, or through instruction/direction of a governmental/health organization, or any combination thereof, according to aspects of the embodiments.

FIG. 3 illustrates a block diagram of thermal data collection and aggregation network (thermal aggregation NW) 300 that can be used to facilitate thermal data collection, aggregation, and analysis according to aspects of the embodiments. Thermal aggregation NW 300 comprises one or more TSSs 100, communications network 302, thermal scan information/data aggregator network servers (TI server) 308, and agency server 312 according to aspects of the embodiments. Communications network 302 can be one or more of cellular communications network 304 and internet 306, among other types of communications networks. By way of non-limiting example, if a user/owner of MED 102 is in their home, MED 102 typically uses a WiFi router connected to a modem to communicate over the internet to communicate with the cellular service provider for the particular MED 102, or, as in this case, the acquired thermal image data can be sent directly to one or more TI servers 308. Agency server 312 can include one or more of network connected servers for one or more government/health organization (e.g., Center for Disease Control (CDC), located in Atlanta, Ga., the World Health Organization (WHO), part of the United Nations (UN), and headquartered in New York, New York, local health departments, state health departments, among other health-related agencies).

According to aspects of the embodiments, each of the one or more TI servers 308 comprises one or more processors 104, memory 106 (internal to, or external to processor 104), and local thermal information analytics application (local TI App) 310 according to aspects of the embodiments. Optionally, TI server 308 can further include database server (database) 311, for storing and manipulating the vast amounts of data that will be collected over time. Such databases 311 include processors, memory, applications, and other software, as those of skill in the art can appreciate. as well as the same or different version of local TI App 310 according to aspects of the embodiments.

According to aspects of the embodiments, each of the one or more agency servers 312 comprises one or more processors 104, memory 106 (internal to, or external to processor 104), and master thermal information analytics application (master TI App) 314 according to aspects of the embodiments. Optionally, agency server 312 can further include database server (database) 311, for storing and manipulating the vast amounts of data that will be collected over time. Such databases 311 include processors, memory, applications, and other software, as those of skill in the art can appreciate. as well as the same or different version of master TI App 314 according to aspects of the embodiments.

According to aspects of the embodiments, master TI App 314 can further process processed thermal image data file 111 in a substantially similar manner as that as TI App 108. In addition, master TI app 314 can direct TI App 108 to perform certain functions it normally would not be able to do on its own; that is, master TI App 314 can direct TI App 108 to acquire personally identifying information in contravention of any PSL settings as described above, and/or privacy laws that might be applicable to the owner of MED 102 based on residence/domiciliary, nationality, or location. Further still, master TI App 314 can provide reports and communications to one or more government officials and/or health organization officials, or the press, according to agreed upon conditions when establishing such health reporting systems. Further functionality of TI App 108 and master TI App 314 are described in greater detail below in regard to method 400.

In operation, relatively soon after acquisition of thermal image data 107 of the user/owner of MED 102, and then stored and ultimately processed by TI App 108 into processed thermal image data file 111, it is transmitted from MED 102 through communications network 302 to TI data server 308, and can be stored and analyzed in memory 106, database 311, or both, or one than the other. Processed thermal image data file 111 can include all, some, or no personal information according to the configuration settings performed in method 200, as well as the thermal image data, which of course indicates a body temperature of the user/owner. Local TI App 310 further processes received processed thermal image data file 111 to produce first aggregated/processed thermal image data 315. Local TI App 310 can strip whatever personal information that is attached to thermal image data 301 in accordance with relevant privacy laws and/or privacy settings as set in PSL in TI App 108, and forward first aggregated/processed thermal image data 315 that includes the basic thermal image data (essentially just body temperature) along with relative (or more exact) location information to agency server 312. Local TI App 310 can also accumulate a substantial amount of first aggregated/processed thermal image data 315 prior to transmitting it to agency server 312, or it can send each first aggregated/processed thermal image data 315 package as it is received and processed by local TI App 310. Local TI App 310 keeps both the originally received processed thermal image data file 111 and first aggregated/processed thermal image data 315 in the event that notification needs to be made to the owner/use of MED 102 in regard to a health crisis, such as an outbreak, epidemic, or pandemic. According to aspects of the embodiments, forwarding first aggregated/processed thermal image data 315 allows health and government officials to monitor relative health of a population without knowing specifically which person may or may not be healthy, thus ensuring, during normal times, privacy that does not necessarily represent a clear and present danger to the overall community.

According to well-established health/medical definitions, an epidemic is a disease that affects a large number of people within a community, population, or region, a pandemic is an epidemic that's spread over multiple countries or continents, an endemic is something that belongs to a particular people or country, and an outbreak is a greater-than-anticipated increase in the number of endemic cases. An outbreak can also be a single case in a new area; if it's not quickly controlled, an outbreak can become an epidemic (See, https://intermountainhealthcare.org/blogs/topics/live-well/2020/04/whats-the-difference-between-a-pandemic-an-epidemic-endemic-and-an-outbreak/)

According to further aspects of the embodiments, the forwarding of accumulated or individual first aggregated/processed thermal image data 315 occurs only when health emergencies are declared by the appropriate governmental authority. According to aspects of the embodiments, the health emergencies can be local, national, or international in nature, and each can have their own set of rules for reporting data to agency server 312. According to further aspects of the embodiments, such rules can be different for different types of health emergencies (i.e., different types of diseases, among other factors). According to further aspects of the embodiments, monitoring of a persons temperature over a period of time can indicate what type of disease her or she may be inflicted with; such prognosis capabilities can be supplemented with the use of health monitor devices 136, 138, discussed and described above. That is, over time, a person can be roughly diagnosed with a certain disease or at least one or more diseases can be indicated by a person's temperature over time, and additional vital sign information can assist in such determinations. Once such a determination has been reached, additional queries can be pushed to the person via text/emails/phone calls by one or more local/national/international health agencies. According to further aspects of the embodiments, the acquisition and tracking over time of an owner's body temperature data can be used by TI App 108 to self-generate warnings to the owner of MED 102 that his/her body temperature is normal or not normal, or heading towards elevated temperatures or returning to more normal body temperatures. Such warnings can also take into account other health information, if available, such as blood pressure, and heart rate, if devices 134, 136 are in use, as described herein.

In such cases when there are bona fide national/international health emergencies, the rules set up in local TI App 310 can be such that no personal information is stripped from first aggregated/processed thermal image data 315 prior to being forwarded to agency server 312. In addition, it can be the case that local TI App 310 can reach out to individual MEDs 102 and reconfigure TI App 108 to include all personal data in each transmission of processed thermal image data file 111, regardless of what the user/owner may have originally configured TI App 108 to report. According to further aspects of the embodiments, it can be the case the use of MED 102 is conditioned upon a user/owner providing personal information such as medical history, contact information, addresses, and the like, as described above when configuring TI App 108. That is, unless the user/owner provides such information, MED 102 cannot be sold and/or put into use.

As briefly discussed above, processed thermal image data file 111 can be transmitted to TI data server 308 communications network 302 and paths A and B. In addition, processed thermal image data file 111 can also be transmitted directly to agency server 312 via communications network 302 and paths A and D to agency server 312. Further still, path C represents communications between TI data server 308 and agency server 312, and can be embodied as communications network 302, or can be some other type of network.

Once processed thermal image data file 111 is received by TI data server 308 and local TI App 310, local TI App 310 begins processing it into first aggregated/processed thermal image data 315. Such processing can entail essentially nothing to complete removal of all identifying personal information other than zip code, as described above. In addition, as local TI App 310 begins receiving enough first aggregated/processed thermal image data 315, it can begin to aggregate and analyze such data. From such aggregation and analyzation, it can be determined which sections of a city or locale are experiencing elevated body temperatures indicating a possible outbreak or more of one or more diseases. According to aspects of the embodiments, such aggregating and analyzing can include all data, such that detailed studies of possible infectious outbreaks can be relatively identified, and once such analysis and aggregation is complete, then data subject to privacy laws or in conformance with the settings previously described can be removed. That is, according to aspects of the embodiments, it can be the case that all personal/private data would necessarily be included in each processed thermal image data file 111 transmission, but then such data is removed in accordance with the PSL settings as set by the owner of MED 102 described above by TI data server 308. As such, important data is always available for governmental and health authorities to access should the need arise.

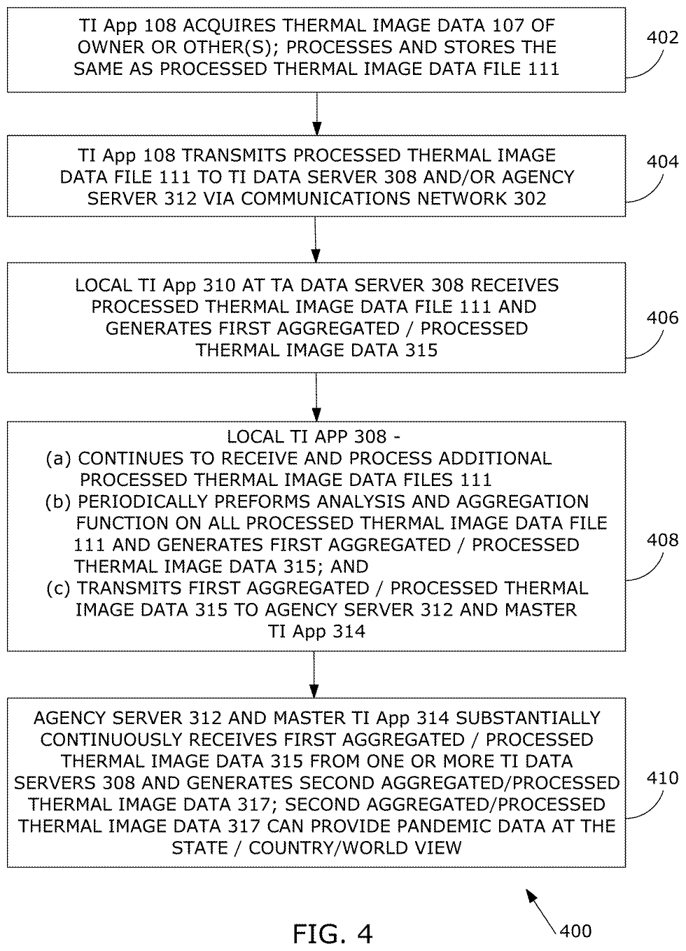

FIG. 4 illustrates a flow chart of a method for performing thermal image data analytics (method 400) according to aspects of the embodiments. The following discussion of method 400 shall focus primarily, initially, on use of TI server 308 and TI App 108 performing any processing, analyzing, and aggregating of processed thermal image data file 111 and first aggregated/processed thermal image data 315 prior to transmitting the same to agency server 312. According to aspects of the embodiments, any and all such processing that occurs in TI server 308 with use of TI App 108 can also occur in agency server 312 and master TI App 314, as discussed above. In addition, master TI app 314 has the ability command TI App 108 to perform certain functions that it normally may not do on its own, such as acquiring personal data, even in contravention of whatever privacy laws might be applicable to the owner of MED 102 depending on residence, nationality, or location.

Method 400 begins with method step 402, in which TI App 108 acquires thermal image data 107 of the owner of MED 102 and processes/stores/converts it to processed thermal image data file 111 in the manner as discussed above in regard to FIGS. 1-3. According to further aspects of the embodiments, TI App 108 can prompt an owner of MED 102 that thermal image data 107 needs to be acquired, or can acquire thermal image data 107 whenever the owner checks their MED 102, or such thermal image data 107 can be acquired randomly, periodically, or a combination of any of such collections methods. Such prompts can be audible, visual, electronic (phone call, text, email, or any combination thereof). According to further aspects of the embodiments, MED 102 and TI App 108 can acquire thermal image data 107 of others (using the forward-facing camera) in substantially any of the aforementioned collection methods (e.g., randomly, periodically, via a reminder, among others). According to aspects of the embodiments, TI App 108 can acquire thermal image data 107 at set times of the day, such as in the morning, afternoon, and evening.

In method step 404, TI App 108 transmits processed thermal image data file 111 to TI data server 308 via communications network 302. In method step 406, local TI App 310 receives processed thermal image data file 111, and generates first processed/aggregated thermal image data 315.

According to aspects of the embodiments, local TI App 310 can remove all personally identifying information except relative or general location in accordance with one or more privacy laws, and/or the PSL as discussed above in regard to FIG. 2, and method 200. That is, it is typically the case that all personally identifying information stored on MED 102 as part of the initialization/setup process be transmitted as part of processed thermal image data file 111. Local TI App 310 can then scrub or remove the data when generating first processed/aggregated thermal image data 315 that, in ordinary times, would be sent to agency server 312 according to aspects of the embodiments.

In method step 408, local TI App 310 continues to receive, and process processed thermal image data files 111 to generate additional first aggregated/processed thermal image data 315. According to aspects of the embodiments, in method step 408, local TI App 310 periodically performs further, more detailed analysis/aggregation of processed thermal image data files 111 and generates one or more reports, which are called first aggregated/processed thermal image data 315.

In method step 410, agency server 312 and master TI App 314 substantially continuously and/or periodically received first aggregated/processed thermal image data 315 from one or more TI data servers 308. Master TI App 314 processes the received first aggregated/processed thermal image data 315 and generates second aggregated/processed thermal image data 317, which can be a more global view of an epidemic or pandemic situation, or not. That is, the accumulation and further processing of a plurality of first aggregated/processed thermal image data 315 from a plurality of TI data serves 308 can provide more detailed information on how one or more infectious diseases might be spreading, or not, and can lead to more detailed data reporting (i.e., reaching back to the owners MED 102 and overriding the PSL he/she may have first set up to allow for all personal/private health data to be transmitted in processed thermal image data file 111), more rules/orders to institute restrictions on movement of people and businesses being open, or, conversely, can release or alleviate, or lift such conditions as the situation improves over time. Method 400 substantially continuously repeats as needed.

According to aspects of the embodiments, local TI App 310 can perform such analysis and generate such reports periodically, or randomly, or according to some schedule based on a certain amount of received data. Such periodicity can be hourly, daily, weekly, depending on the number of received thermal image data received, or the number based on geographical location, and so on.

According to aspects of the embodiments, any one of TI App 108, local TI App 310, and master TI App 314 and can perform basic health analysis based on one or more of the thermal image data they acquire in regard to owner of MED 102. In particular, according to further aspects of the embodiments, TI App 108 can perform basic health analysis based on one or more of processed thermal image data files 111 it acquires in regard to owner of MED 102, and other health monitoring data if devices 136, 138 are also used. Such health analysis can include a rough diagnosis of one or more ailments that a person could be afflicted with. Such ailments can be based on one or more of a body temperature, blood pressure reading, and heart rate of the owner of MED 102. In such cases, a notification can be sent to the owner, as well as local health and government authorities, including one or more physicians of the owner of MED 102. According to further aspects of the embodiments, such communications to one or more of the health authorities and government officials of an ailment of the owner of MED 102 can bypass privacy laws based on location, domiciliary, and/or national origin—that is, the generated health reports can be sent regardless of privacy settings and in contravention of the wishes/desires of the owner of MED 102. Communications to the owner of MED 102 can include one or more of an automated or personal phone call, text, email, or physical correspondence (e.g., U.S. Postal Service). Communications to the health and government officials can be linked with the health data of other close-by users, so that a complete health status of a specific geographical area can be determined. According to further aspects of the embodiments, such compiled health information may or may not be reported to the owner of MED 102, and can also be transmitted to agency server 312 and master TI App 314 for further analysis and processing.

According to aspects of the embodiments, many different functions have been described in regard to TI App 108, local TI App 308, and master TI App 314; according to further aspects of the embodiments, any one or more or all of those functions can be substantially similar performed by any of TI App 108, local TI App 308, and master TI App 314, either in parallel or separately.

According to aspects of the embodiments, as described herein in regard to FIGS. 1-4, processed thermal image data files 111 can be transmitted directly from MED 102 to TI data server 308, and then processed thermal image data files 111 and first aggregated/processed thermal image data 315 can be transmitted from TI data server 308, along with additional analysis, to agency server 312 for further processing and dissemination by master TI app 314 according to aspects of the embodiments.

According to aspects of the embodiments, as described herein in regard to FIGS. 1-4, processed thermal image data files 111 can also be transmitted directly from MED 102 to agency server 312 for further processing and dissemination by master TI app 314 according to aspects of the embodiments. In this case, the processing that was performed by local TI App 310 can be performed by master TI App 314.

According to aspects of the embodiments, as described herein in regard to FIGS. 1-4, processed thermal image data files 111 can be transmitted from MED 102 to both TS data server 308 and agency server 312 for substantially concurrent (or not) processing by both local TI App 310 and master TI App 314 according to aspects of the embodiments. In this case, processing is performed by both local TI App 310 and master TI App 314.

According to aspects of the embodiments, any and all communications received by TI data server 308 can also be received by agency server 314 either in parallel or in place of TI data server 308.

Aspects of the embodiments, including TI App 108, local TI App 310, and master TI App 314 working separately or in combination, can monitor and track viruses that spread around the globe. If any of TI App 108, local TI App 310, and master TI App 314 and the other aspects of the embodiments detect significant amounts of people with elevated body temperatures in a particular area, various health organizations, including the WHO and/or CDC, among others, can be alerted to take measures to prevent global pandemics. Such measures can include personal monitoring of such infected people by physicians and other health care workers. Such personal monitoring can include determining if and what type of virus the people might be infected with. According to aspects of the embodiments, the systems, methods, and modes described herein can facilitate notification of people that might be in close proximity to the infected people. According to further aspects of the embodiments, such measure to prevent global pandemics can further include travel restrictions of infected people, those that might be infected, those that have been in close contact with infected people, and such travel restrictions can include travel by everyone, not just infected people, to anyplace in the world.

According to further aspects of the embodiments, the systems, methods, and modes described herein can also track and monitor people that are showing signs of infection with an elevated body temperature only if there have been one or more indications of a dangerous virus that could threaten millions of people and global economies. Such indications can include clusters of people being admitted to the hospitals or reports of such illnesses by physicians, such as primary care physicians. According to aspects of the embodiments, the systems, methods, and modes described herein can alert people that might have elevated body temperatures automatically, with or without contacting government and/or health officials of such potential illnesses. According to aspects of the embodiments, such alerts can include notifications that such potentially infected people should begin to wear protective gear, such as face masks, shields, and the like, and begin processes of self-quarantining.

According to aspects of the embodiments, the systems, methods, and modes described herein can automatically alert the physician or local hospital/emergency room/walk-in clinic of the owner of MED 102 should elevated body temperatures be indicated by thermal image data 107, and wherein such elevated body temperatures might indicate an illness such as a viral contagious illness.

According to aspects of the embodiments, the systems, methods, and modes described herein can acquire one or more thermal images of other people beside the owner of MED 102. That is, forward facing internal camera 126 can acquire thermal image data 107 of whomever happens to be in close proximity to MED 102. Such acquired thermal image data 107 of others can be acquired whenever other people are detected within range of forward-facing camera 126, or it can be done at specific intervals, or randomly, or in any combination thereof. According to aspects of the embodiments, such acquired data can provide a substantial amount of data in regard to infection levels in a wide geographical area. In addition, privacy laws are not a concern, as no one person could be identified from the thermal image—the only data that is transmitted is the thermal data.

According to aspects of the embodiments, the systems, methods, and modes described herein can, after acquiring such thermal image data 107 and processing the same, can allow for health and government authorities to send alerts to the owner of MED 102 based on his/her own data, or on the processed results of processed thermal image data files 111 from a plurality of others. Such alerts can include warnings about possibly infected people, or that the owner of MED 102 is possibly infected. For example, if the acquired thermal image data 107 of other people showed a significant amount of people with elevated body temperatures, or even a single person with an elevated body temperature, one or more of TI data server 308 and agency server 312 and respective applications local TI App 310 and master TI App 314 can alert the owner of MED 102 that one or more people the owner might have been in close proximity to might be sick, and to take appropriate precautions. According to further aspects of the embodiments, additional information can also be pushed to the owner of MED 102, including, but not limited to, natural disaster information, other types of emergency information, and the like.

Network 302 can be one or more different or separate networks, and can provide wired or wireless communications between nodes, such as MED 102, TI data server 308, and agency server 312. Each of the aforementioned computing devices 102, 308, 312 can each include a display device, such as a touch enabled display component, and a monitor, among others, to provide access to any of TI App 108, local TI App 310, and master TI App 314 for the users through a web browser (thin client) or a local client application (thick client). Servers 308, 312 can include a desktop computer, a laptop computer, a tablet, a handheld device, a vehicle mount computer, an embedded computer system, a smart phone, and a wearable computer, among other computing devices, for example.

While computing network environment 300 as illustrated in FIG. 3 has been described with specific components including servers 308, 312, and MED 102, aspects of the embodiments are not limited to these components or system configurations and can be implemented with other system configuration employing fewer or additional components.

As discussed above, there are currently no existing systems that are capable of identifying the origins of an epidemic or pandemic quickly and efficiently. It can be appreciated by those of skill in the art that application of detecting and tracking infected individuals is far from trivial. There are no known systems that can affect such detection, tracking and notification. According to aspects of the embodiments, the systems, methods and modes as described herein (e.g., MED 102, servers 308, 312, and TI App 108, local TI App 310, and master TI App 314) improve the accuracy of the health data through the use of MEDs 102 and acquired thermal image data 107 of the owner of MED 102 and others in close proximity to the owner of MED 102.

According to further aspects of the embodiments, the systems and methods described herein, and as shown in regard to the Figures as well, can incorporate additional features and functions, which are related to data management, security protection. Such information management can use “Big Data” and “Internet of Things” methods and systems (which can also be referred to as data analytics (“Data analysis is a process of inspecting, cleansing, transforming and modeling data with the goal of discovering useful information, informing conclusion and supporting decision-making”), among other features and functions.

FIG. 5 illustrates a block diagram of the major components of a computer device, personal computer, server, laptop, and/or personal electronic device (herein after, “computer device”) suitable for use to implement one or more of the methods shown in regard to FIG. 2 or 4, among others, for generating, distributing, and processing thermal images of one or more people using a mobile electronic device and one or more computer devices using the thermal image applications according to aspects of the embodiments.

FIG. 5 illustrates a block diagram of the major components of a computer device, PC, server, laptop, and/or Devices 308 and 312, and mobile device 102 (herein after, “computer device”) suitable for use to implement methods 200 and 400, among others, for personal thermal image data collection and management according to aspects of the embodiments (although computer device is meant to describe each of the aforementioned devices, the numbering of components has been kept consistent with this Figure in order to make the discussion clearer; that is, while a processor 104 has been used and described in reference to FIG. 1, the processor in FIG. 5 is defined as processor 506, even though the processors are substantially similar, and the same applies for other components as well). The computer device comprises, among other items, shell/box 501, integrated display/touch-screen 502 (though not used in every application of the computer device), internal data/command bus (bus) 504, processor board/PC internal memory (internal memory) 508, and one or more processors 506 with processor internal memory 508 (which can be typically read only memory (ROM) and/or random access memory (RAM)). Those of ordinary skill in the art can appreciate that in modern computer device systems, parallel processing is becoming increasingly prevalent, and whereas a single processor would have been used in the past to implement many or at least several functions, it is more common currently to have a single dedicated processor for certain functions (e.g., digital signal processors) and therefore could be several processors, acting in serial and/or parallel, as required by the specific application.

The computer device further comprises multiple input/output ports, such as universal serial bus (USB) ports 510, Ethernet ports 511, and video graphics array (VGA) ports/high definition multimedia interface (HDMI) ports 522, among other types. Further, the computer device includes externally accessible drives such as compact disk (CD)/digital versatile disk (DVD) read/write (RW) (CD/DVD/RW) drive 512, and floppy diskette drive 514 (though less used currently, some computer devices still include this type of interface). The computer device still further includes wireless communication apparatus, such as one or more of the following: Wi-Fi transceiver 542, BlueTooth (BT) transceiver 544, near field communications (NFC) transceiver 546, third generation (3G)/fourth Generation (4G)/long term evolution (LTE)/fifth generation (5G) transceiver (cellular transceiver) 548, communications satellite/global positioning system (satellite) transceiver 550, and antenna 552.

Internal memory 508 itself can comprise hard disk drive (HDD) 516 (these can include conventional magnetic storage media, but, as is becoming increasingly more prevalent, can include flash drive memory 534, among other types), ROM 518 (these can include electrically erasable programmable ROM (EEPROMs), ultra-violet erasable PROMs (UVPROMs), among other types), and RAM 520. USB port 510d is usable with flash drive memory 534, and CD/DVD/RW drive 512 is usable with CD/DVD diskettes (CD/DVD) 536 (which can be both read and write-able). Usable with floppy diskette drive 514 are floppy diskettes 538. External memory storage device 524 can be used to store data and programs external to box 501 of the computer device, and can itself comprise another hard disk drive 516a, flash drive memory 534, among other types of memory storage. External memory storage device 524 is connectable to the computer device via USB cable 556, among other types of data transfer cables.

Each of the memory storage devices, or the memory storage media (506, 516, 518, 520, 524, 534, 536, and 538, among others), can contain parts or components, or in its entirety, executable software programming code or application that has been termed TI App 108, Local TI App 310, and master TI App 314 according to aspects of the embodiments, which can implement part or all of the portions of methods 200, and 400, among other methods not shown, described herein.

In addition to the above described components, the computer device also comprises keyboard 528, external display 526, printer/scanner/fax machine 560, and mouse 530 (although not technically part of the computer device, the peripheral components as shown in FIG. 5 (524, 526, 528, 530, 534, 536, 538, 556, 558, 560, and 562) are adapted for use with the computer device that for purposes of this discussion they shall be considered as being part of the computer device). Other cable types that can be used with the computer device include RS 232, among others, not shown, that can be used for one or more of the connections between the computer device and the peripheral components described herein. Keyboard 528, and mouse 530 are connectable to the computer device via USB cable 562, and external display 526 is connectible to the computer device via VGA cable/HDMI cable 562. The computer device is connectible to network 302 via Ethernet port 511 and Ethernet cable 558 via a router and modulator-demodulator (MODEM) and internet service provider, none of which are shown in FIG. 5. All of the immediately aforementioned components (524, 526, 528, 530, 534, 536, 538, 556, 558, 560, and 562) are known to those of ordinary skill in the art, and this description includes all known and future variants of these types of devices.

External display 526 can be any type of currently available display or presentation screen, such as liquid crystal displays (LCDs), light emitting diode displays (LEDs), plasma displays, cathode ray tubes (CRTs), among others (including touch screen displays). In addition to the user interface mechanism such as mouse 530, the computer device can further include a microphone, touch pad, joy stick, touch screen, voice-recognition system, among other inter-active inter-communicative devices/programs, which can be used to enter data and voice, and which all of are currently available and thus a detailed discussion thereof has been omitted in fulfillment of the dual purposes of clarity and brevity.

As mentioned above, the computer device further comprises a plurality of wireless transceiver devices, such as Wi-Fi transceiver 542, BT transceiver 544, NFC transceiver 546, cellular transceiver 548, satellite transceiver 550, and antenna 552. While each of Wi-Fi transceiver 542, BT transceiver 544, NFC transceiver 546, cellular transceiver 548, and satellite transceiver 550 has their own specialized functions, each can also be used for other types of communications, such as accessing a cellular service provider (not shown), accessing network 302 (which can include the Internet), texting, emailing, among other types communications and data/voice transfers/exchanges, as known to those of skill in the art. Each of Wi-Fi transceiver 542, BT transceiver 544, NFC transceiver 546, cellular transceiver 548, satellite transceiver 550 includes a transmitting and receiving device, and a specialized antenna, although in some instances, one antenna 552 can be shared by one or more of Wi-Fi transceiver 542, BT transceiver 544, NFC transceiver 546, cellular transceiver 548, and satellite transceiver 550. Alternatively, one or more of Wi-Fi transceiver 542, BT transceiver 544, NFC transceiver 546, cellular transceiver 548, and satellite transceiver 550 will have a specialized antenna, such as satellite transceiver 550 to which is electrically connected at least one antenna 552.

In addition, the computer device can access network 302 (which can be the Internet), either through a hard wired connection such as Ethernet port 811 as described above, or wirelessly via Wi-Fi transceiver 542, cellular transceiver 548 and/or satellite transceiver 550 (and their respective antennas) according to aspects of the embodiments. The computer device can also be part of a larger network configuration as in a global area network (GAN) (e.g., internet), which ultimately allows connection to various landlines.

According to further aspects of the embodiments, integrated touch screen display 502, keyboard 528, mouse 530, and external display 526 (if in the form of a touch screen), can provide a means for a user to enter commands, data, digital, and analog information into the computer device. Integrated and external displays 502, 526 can be used to show visual representations of acquired data, and the status of applications that can be running, among other things.

Bus 504 provides a data/command pathway for items such as: the transfer and storage of data/commands between processor 508, Wi-Fi transceiver 542, BT transceiver 544, NFC transceiver 546, cellular transceiver 548, satellite transceiver 550, integrated display 502, USB port 510, Ethernet port 511, VGA/HDMI port 522, CD/DVD/RW drive 512, floppy diskette drive 514, and internal memory 532. Through bus 504, data can be accessed that is stored in internal memory 532. Processor 508 can send information for visual display to either or both of integrated and external displays 502, 526, and the user can send commands to the system operating TI App 108, local TI App 310, and/or master TI App 314 that might reside in processor internal memory 506 of processor 508, or any of the other memory devices (536, 538, 516, 518, and 520).

The computer device, and either processor internal memory 506 or internal memory 532, can be used to implement one or more, or any combination thereof, methods 200 and 400, among others, for personal thermal image data collection and management using one or more computer devices and mobile electronic devices according to aspects of the embodiments. Hardware, firmware, software, or a combination thereof can be used to perform the various steps and operations described herein. According to aspects of the embodiments, TI App 108, local TI App 310, and master TI App 314 for carrying out the above discussed steps can be stored and distributed on multi-media storage devices such as devices 516, 518, 520, 534, 536 and/or 538 (described above) or other form of media capable of portably storing information. Storage media 534, 536 and/or 538 can be inserted into, and read by devices such as USB port 510, CD/DVD/RW drive 512, and floppy disk drive 514, respectively.

As also will be appreciated by one skilled in the art, the various functional aspects of the aspects of the embodiments can be embodied in a wireless communication device, a telecommunication network, or as a method or in a computer program product. Accordingly, aspects of embodiments can take the form of an entirely hardware embodiment or an embodiment combining hardware and software aspects. Further, the aspects of embodiments can take the form of a computer program product stored on a computer-readable storage medium having computer-readable instructions embodied in the medium. Any suitable computer-readable medium can be utilized, including hard disks, CD-ROMs, DVDs, optical storage devices, or magnetic storage devices such a floppy disk or magnetic tape. Other non-limiting examples of computer-readable media include flash-type memories or other known types of memories.

Further, those of ordinary skill in the art in the field of the aspects of the embodiments can appreciate that such functionality can be designed into various types of circuitry, including, but not limited to field programmable gate array structures (FPGAs), application specific integrated circuitry (ASICs), microprocessor based systems, among other types. A detailed discussion of the various types of physical circuit implementations does not substantively aid in an understanding of the aspects of the embodiments, and as such has been omitted for the dual purposes of brevity and clarity. However, the systems and methods discussed herein can be implemented as discussed and can further include programmable devices.

Such programmable devices and/or other types of circuitry as previously discussed can include a processing unit, a system memory, and a system bus that couples various system components including the system memory to the processing unit. The system bus can be any of several types of bus structures including a memory bus or memory controller, a peripheral bus, and a local bus using any of a variety of bus architectures. Furthermore, various types of computer readable media can be used to store programmable instructions. Computer readable media can be any available media that can be accessed by the processing unit. By way of example, and not limitation, computer readable media can comprise computer storage media and communication media. Computer storage media includes volatile and nonvolatile as well as removable and non-removable media implemented in any method or technology for storage of information such as computer readable instructions, data structures, program modules or other data. Computer storage media includes, but is not limited to, RAM, ROM, EEPROM, flash memory or other memory technology, CD-ROMs, DVDs or other optical disk storage, magnetic cassettes, magnetic tape, magnetic disk storage or other magnetic storage devices, or any other medium which can be used to store the desired information and which can be accessed by the processing unit. Communication media can embody computer readable instructions, data structures, program modules or other data in a modulated data signal such as a carrier wave or other transport mechanism and can include any suitable information delivery media.

The system memory can include computer storage media in the form of volatile and/or nonvolatile memory such as ROM and/or RAM. A basic input/output system (BIOS), containing the basic routines that help to transfer information between elements connected to and between the processor, such as during start-up, can be stored in memory. The memory can also contain data and/or program modules that are immediately accessible to and/or presently being operated on by the processing unit. By way of non-limiting example, the memory can also include an operating system, application programs, other program modules, and program data.

The processor can also include other removable/non-removable and volatile/nonvolatile computer storage media. For example, the processor can access a hard disk drive that reads from or writes to non-removable, nonvolatile magnetic media, a magnetic disk drive that reads from or writes to a removable, nonvolatile magnetic disk, and/or an optical disk drive that reads from or writes to a removable, nonvolatile optical disk, such as a CD-ROM or other optical media. Other removable/non-removable, volatile/nonvolatile computer storage media that can be used in the operating environment include, but are not limited to, magnetic tape cassettes, flash memory cards, digital versatile disks, digital video tape, solid state RAM, solid state ROM and the like. A hard disk drive can be connected to the system bus through a non-removable memory interface such as an interface, and a magnetic disk drive or optical disk drive can be connected to the system bus by a removable memory interface, such as an interface.

Aspects of the embodiments discussed herein can also be embodied as computer-readable codes on a computer-readable medium. The computer-readable medium can include a computer-readable recording medium and a computer-readable transmission medium. The computer-readable recording medium is any data storage device that can store data which can be thereafter read by a computer system. Examples of the computer-readable recording medium include ROM, RAM, CD-ROMs and generally optical data storage devices, magnetic tapes, flash drives, and floppy disks. The computer-readable recording medium can also be distributed over network coupled computer systems so that the computer-readable code is stored and executed in a distributed fashion. The computer-readable transmission medium can transmit carrier waves or signals (e.g., wired, or wireless data transmission through the Internet). Also, functional programs, codes, and code segments to, when implemented in suitable electronic hardware, accomplish or support exercising certain elements of the appended claims can be readily construed by programmers skilled in the art to which the aspects of the embodiments pertains.

The disclosed aspects of the embodiments provide a system and method for personal thermal image data collection and management on one or more computer devices and mobile electronic devices. It should be understood that this description is not intended to limit aspects of the embodiments. On the contrary, aspects of the embodiments are intended to cover alternatives, modifications, and equivalents, which are included in the spirit and scope of the aspects of the embodiments as defined by the appended claims. Further, in the detailed description of the aspects of the embodiments, numerous specific details are set forth to provide a comprehensive understanding of the claimed aspects of the embodiments. However, one skilled in the art would understand that various aspects of the embodiments can be practiced without such specific details.

FIG. 6 illustrates network system 600 within which the system and method for personal thermal image data collection and management on one or more computer devices and mobile electronic devices can be implemented according to aspects of the embodiments. Much of the infrastructure of network system 600 shown in FIG. 6 is or should be known to those of skill in the art, so, in fulfillment of the dual purposes of clarity and brevity, a detailed discussion thereof shall be omitted.

According to aspects of the embodiments, a user of the above described system and method can store TI App 108 on MED 102, local TI App on TI data server 308, and master TI App 314 on agency server 312. MED 102 can include, but are not limited to, so-called smart phones, tablets, personal digital assistants (PDAs), notebook and laptop computers, and essentially any device that can access the internet and/or cellular phone service or can facilitate transfer of the same type of data in either a wired or wireless manner.

MED 102 can access cellular service provider 614, either through a wireless connection (cellular tower 620) or via a wireless/wired interconnection (a “Wi-Fi” system that comprises, e.g., modulator/demodulator (modem) 608, wireless router 610, internet service provider (ISP) 606, and internet 622). Further, PED 102 can include NFC, “Wi-Fi,” and Bluetooth (BT) communications capabilities as well, all of which are known to those of skill in the art. To that end, network system 106 further includes, as many homes (and businesses) do, one or more PEDs 102 that can be connected to wireless router 610 via a wired connection (e.g., modem 608) or via a wireless connection (e.g., Bluetooth). Modem 608 can be connected to ISP 606 to provide internet-based communications in the appropriate format to end users (e.g., PED 102), and which takes signals from the end users and forwards them to ISP 606.

PEDs 102 can also access global positioning system (GPS) satellite 628, which is controlled by GPS station 624, to obtain positioning information (which can be useful for different aspects of the embodiments), or PEDs 102 can obtain positioning information via cellular service provider 614 using cellular tower(s) 620 according to one or more methods of position determination. Some PEDs 602 can also access communication satellites 618 and their respective satellite communication systems control stations 626 (the satellite in FIG. 6 is shown common to both communications and GPS functions) for near-universal communications capabilities, albeit at a much higher cost than convention “terrestrial” cellular services. MED 102 can also obtain positioning information when near or internal to a building (or arena/stadium) through the use of one or more of NFC/BT devices. FIG. 6 also illustrates other components of network 600 such as plain old telephone service (POTS) provider 612.

According to further aspects of the embodiments, and as described above, network 600 also contains servers/devices 102, 308, and 312 that can include TI App 108, local TI App 312, and master TI App 314, respectively, wherein one or more processors, using currently available technology, such as memory, data and instruction buses, and other electronic devices, can store and implement code that can implement the system and method for tracking thermal images of a user and one or more other people in vicinity of the user on a computer device according to aspects of the embodiments.

As described above, one or more encoding processes are discussed specifically in reference to FIGS. 2 and 4, although such delineation is not meant to be, and should not be taken in a limiting manner, as additional methods according to aspects of the embodiments have been described herein. The encoding processes as described are not meant to limit the aspects of the embodiments, or to suggest that the aspects of the embodiments should be implemented following the encoding processes. The purpose of the encoding processes as described is to facilitate the understanding of one or more aspects of the embodiments and to provide the reader with one or many possible implementations of the processed discussed herein. FIGS. 2 and 4 illustrate respective flowcharts of various steps performed during the encoding process, but such encoding processes are not limited thereto. The steps of FIGS. 2 and 4 are not intended to completely describe the encoding processes but only to illustrate some of the aspects discussed above.

This application may contain material that is subject to copyright, mask work, and/or other intellectual property protection. The respective owners of such intellectual property have no objection to the facsimile reproduction of the disclosure by anyone as it appears in published Patent Office file/records, but otherwise reserve all rights.

The disclosed embodiments provide a system, software, and a method for tracking thermal images of a user and one or more other people in vicinity of the user on a computer device. It should be understood that this description is not intended to limit the embodiments. On the contrary, the embodiments are intended to cover alternatives, modifications, and equivalents, which are included in the spirit and scope of the embodiments as defined by the appended claims. Further, in the detailed description of the embodiments, numerous specific details are set forth to provide a comprehensive understanding of the claimed embodiments. However, one skilled in the art would understand that various embodiments may be practiced without such specific details.

Although the features and elements of aspects of the embodiments are described being in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

The above-described embodiments are intended to be illustrative in all respects, rather than restrictive, of the embodiments. Thus, the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

### INDUSTRIAL APPLICABILITY

To solve the aforementioned problems, the aspects of the embodiments are directed towards systems, methods, and modes for a computer-based dynamic content generation application that facilitates document creation through the substantially seamless synthesis of information from multiple reference files and file types to edit text/content within one integrated space.

### Alternate Embodiments

Alternate embodiments may be devised without departing from the spirit or the scope of the different aspects of the embodiments.

What is claimed is:

1. A thermal image data acquisition device (100), comprising:
at least one wireless transceiver device (112, 114, 116), adapted to transmit and receive digital data;
at least one infrared camera device (126, 134, 132), adapted to acquire one or more thermal images of an owner of the thermal image data acquisition device, wherein each of the one or more thermal images records an internal body temperature of the owner;
a processor (104) communicatively coupled to the at least one wireless transceiver device and the at least one infrared camera device; and
a memory (106) operatively connected to the processor, wherein the memory stores computer-executable instructions that, when executed by the processor, causes the at least one processor to execute a method that comprises:
acquiring, via the at least one infrared camera device, the one or more thermal images within a thermal imaging application (TI App) (108) executing on the processor; and
processing the acquired one or more thermal images in accordance with one or more processing instructions, wherein the one or more processing instructions generates one or more processed thermal image files;
transmitting each of the one or more processed thermal image files of the owner; and
making a determination as to whether a pandemic situation exists within a specific geographical area based on the recorded internal body temperature of the owner as stored in the one or more processed thermal data image files of the owner.

2. The thermal image data acquisition device according to claim 1, wherein
the at least one infrared camera device is an internal infrared camera device that is substantially completely contained within a body of the thermal data image acquisition device.

3. The thermal image data acquisition device according to claim 1, wherein
the at least one infrared camera device is an external infrared camera device that is electrically connected to the thermal data image acquisition device through a communications port.

4. The thermal image data acquisition device according to claim 1, further comprising:
a health monitor adapted to be worn by a non-infant person, monitor one or more vital signs of the non-infant person, generate one or more vital signs information of a non-infant person, and which is further adapted to transmit the one or more vital sign information to the thermal data acquisition device, and wherein
the one or more vital signs information provides information about a health state of the non-infant person.

5. The thermal image data acquisition device according to claim 4, wherein
the one or more vital signs can include a heart rate, a pulse oximeter reading, and a blood pressure.

6. The thermal image data acquisition device according to claim 1, wherein the step of processing the acquired one or more thermal images by the method that is executed by the processor further comprises:
applying a privacy setting level to the thermal image file of the owner.

7. The thermal image data acquisition device according to claim 6, wherein the privacy setting level comprises:
a set of rules established by the owner of the thermal image data acquisition device that determines what types of personal data of the owner can be stored with the acquired thermal image data file, and wherein
the set of rules is stored in the thermal image data acquisition device and accessed by the thermal imaging application when processing the acquired one or more thermal images of the owner, and wherein
processing occurs in accordance with the set of rules established by the owner.

8. The thermal image data acquisition device according to claim 7, wherein
the set of rules established by the owner of the thermal image data acquisition device can be ignored by the thermal imaging application when directed by either or both of a government agency and health agency.

9. The thermal image data acquisition device according to claim 1, wherein the step of making a determination by the method that is executed by the processor comprises:
applying statistical analysis of the processed thermal image data files of a fixed, predetermined number of people that live within a first fixed radius of the owner of the thermal data image acquisition device to determine whether a first threshold number of people have elevated body temperatures.

10. A thermal image data acquisition device (100), comprising:
at least one wireless transceiver device (112, 114, 116), adapted to transmit and receive digital data;
at least one infrared camera device (126, 134, 132), adapted to acquire one or more thermal images of one or more people in the vicinity of an owner of the thermal image data acquisition device, wherein each of the one or more thermal images records an internal body temperature of the one or more people;
a processor (104) communicatively coupled to the at least one wireless transceiver device and the at least one infrared camera device; and
a memory (106) operatively connected to the processor, wherein the memory stores computer-executable instructions that, when executed by the processor, causes the at least one processor to execute a method that comprises:
acquiring, via the at least one infrared camera device, the one or more thermal images within a thermal imaging application (TI App) (108) executing on the processor; and
processing the acquired one or more thermal images in accordance with one or more processing instructions, wherein the one or more processing instructions generates one or more processed thermal data image files;
transmitting each of the one or more processed thermal image files of the one or more people; and
making a determination as to whether a pandemic situation exists within a specific geographical area based on the recorded internal body temperature of the one or more people as stored in the one or more processed thermal image files of the owner.

11. The thermal image data acquisition device according to claim 10, wherein
the at least one infrared camera device is an internal infrared camera device that is substantially completely contained within a body of the thermal data image acquisition device.

12. The thermal image data acquisition device according to claim 10, wherein
the at least one infrared camera device is an external infrared camera device that is electrically connected to the thermal data image acquisition device through a communications port.

13. The thermal image data acquisition device according to claim 10, further comprising:
a health monitor adapted to be worn by a non-infant person, monitor one or more vital signs of the non-infant person, generate one or more vital signs information of a non-infant person, and which is further adapted to transmit the one or more vital sign information to the thermal data acquisition device, and wherein
the one or more vital signs information provides information about a health state of the non-infant person.

14. The thermal image data acquisition device according to claim 13, wherein
the one or more vital signs can include a heart rate, a pulse oximeter reading, and a blood pressure.

15. The thermal image data acquisition device according to claim 10, wherein the method that is executed by the processor further comprises:
obtaining one or more thermal images of the owner of the thermal image data acquisition device.

16. The thermal image data acquisition device according to claim 15, wherein the step of processing the acquired one or more thermal images by the method that is executed by the processor comprises:
applying a privacy setting level to the one or more thermal images of the owner.

17. The thermal image data acquisition device according to claim 16, wherein the privacy setting level comprises:
a set of rules established by the owner of the thermal image data acquisition device that determines what types of personal data of the owner can be stored with the acquired thermal image data, and wherein
the set of rules is stored in the thermal image data acquisition device and accessed by the thermal imaging application when processing the acquired one or more thermal images of the owner, and wherein
processing occurs in accordance with the set of rules established by the owner.

18. The thermal image data acquisition device according to claim 17, wherein
the set of rules established by the owner of the thermal image data acquisition device can be ignored by the thermal imaging application when directed by either or both of a government agency and health agency.

19. The thermal image data acquisition device according to claim 10, wherein the step of making a determination by the method that is executed by the processor comprises:
applying statistical analysis of the processed thermal image data files of a fixed, predetermined number of people that live within a first fixed radius of the owner of the thermal data image acquisition device to determine whether a first threshold number of people have elevated body temperatures.

20. A thermal image data acquisition system, comprising:
one or more thermal image data acquisition devices (100), wherein each of the thermal data acquisition device comprises:
at least one wireless transceiver device (112, 114, 116), adapted to transmit and receive digital data;
at least one infrared camera device (126, 134, 132), adapted to acquire one or more thermal images of an owner of the thermal image data acquisition device, wherein each of the one or more thermal images records an internal body temperature of the owner;
a processor (104) communicatively coupled to the at least one wireless transceiver device and the at least one infrared camera device; and
a memory (106) operatively connected to the processor, wherein the memory stores computer-executable instructions that, when executed by the processor, causes the at least one processor to execute a method that comprises:
acquiring, via the at least one infrared camera device, the one or more thermal images within a thermal imaging application (TI App) (108) executing on the processor; and
processing the acquired one or more thermal images in accordance with one or more processing instructions, wherein the one or more processing instructions generates one or more processed thermal data image files; and
transmitting each of the one or more processed thermal data image files of the owner;
one or more thermal image data collection servers (308), wherein each of the one or more thermal image data collection servers comprises:
at least one communications transceiver device, adapted to transmit and receive digital data;
at least one second processor (104) communicatively coupled to the at least one communications transceiver device; and
a second memory (106) operatively connected to the at least one second processor, wherein the second memory stores computer-executable instructions that, when executed by the at least one second processor, causes the at least one second processor to execute a method that comprises:
receiving, via the at least one communications transceiver device, the transmitted one or more processed thermal data images files within a second thermal imaging application (local TI App) (310) executing on the at least one second processor; and
processing the received one or more thermal data image files in accordance with one or more processing instructions, wherein the one or more processing instructions generates one or more first aggregated/processed thermal image files (315); and
transmitting each of the one or more first aggregated/processed thermal image data files;
one or more agency thermal image data collection servers (312), wherein each of the one or more agency thermal image data collection servers comprises:
at least one communications transceiver device, adapted to transmit and receive digital data;
at least one third processor (104) communicatively coupled to the at least one communications transceiver device; and
a third memory (106) operatively connected to the at least one third processor, wherein the third memory stores computer-executable instructions that, when executed by the at least one third processor, causes the at least one third processor to execute a method that comprises:
receiving, via the at least one communications transceiver device, the transmitted one or more first aggregated/processed thermal data images files within a third thermal imaging application (master TI App) (314) executing on the at least one third processor; and
processing the received one or more first aggregated/processed thermal data image files in accordance with one or more processing instructions, wherein the one or more processing instructions generates one or more second aggregated/processed thermal image files (317); and
making a determination as to whether a pandemic situation exists within a specific geographical area based on the recorded internal body temperature of the owner as stored in the one or more processed thermal data image files of the owner.
